SEQUENCE LISTING (50 sequences)

SEQ ID NO: 1 - protein, 10 aa, synthetic construct
  REGION 1..10: note = Nanobody
GFTFDDYAMG

SEQ ID NO: 2 - protein, 10 aa, synthetic construct
  REGION 1..10: note = Nanobody
ISWSGGNTYY

SEQ ID NO: 3 - protein, 23 aa, synthetic construct
  REGION 1..23: note = Nanobody
AAKPRRTYYSGSDYYTSPYEYDY

SEQ ID NO: 4 - protein, 10 aa, synthetic construct
  REGION 1..10: note = Nanobody
GRTFNSFNMA

SEQ ID NO: 5 - protein, 10 aa, synthetic construct
  REGION 1..10: note = Nanobody
IMWSGTHTRY

SEQ ID NO: 6 - protein, 16 aa, synthetic construct
  REGION 1..16: note = Nanobody
AGQIYGDYFKESNMQY

SEQ ID NO: 7 - protein, 10 aa, synthetic construct
  REGION 1..10: note = Nanobody
GRTFNSFNMA

SEQ ID NO: 8 - protein, 10 aa, synthetic construct
  REGION 1..10: note = Nanobody
IMWSGTHTRY

SEQ ID NO: 9 - protein, 16 aa, synthetic construct
  REGION 1..16: note = Nanobody
AGQIYGDYLKESNMQY

SEQ ID NO: 10 - protein, 10 aa, synthetic construct
  REGION 1..10: note = Nanobody
GRTFSSFNMA

SEQ ID NO: 11 - protein, 10 aa, synthetic construct
  REGION 1..10: note = Nanobody
IMWSGTHTRY

SEQ ID NO: 12 - protein, 16 aa, synthetic construct
  REGION 1..16: note = Nanobody
AAQIYGDYFKESGMQY

SEQ ID NO: 13 - protein, 10 aa, synthetic construct
  REGION 1..10: note = Nanobody
GRTFRYYAMG

SEQ ID NO: 14 - protein, 10 aa, synthetic construct
  REGION 1..10: note = Nanobody
INISGSNTDY

SEQ ID NO: 15 - protein, 12 aa, synthetic construct
  REGION 1..12: note = Nanobody
AANRRGPNDYEY

SEQ ID NO: 16 - protein, 10 aa, synthetic construct
  REGION 1..10: note = Nanobody
GRTFRYYAMG

SEQ ID NO: 17 - protein, 10 aa, synthetic construct
  REGION 1..10: note = Nanobody
INISGSNTDY

SEQ ID NO: 18 - protein, 12 aa, synthetic construct
  REGION 1..12: note = Nanobody
AANRRGRNDYEY

SEQ ID NO: 19 - protein, 10 aa, synthetic construct
  REGION 1..10: note = Nanobody
GRTFRYYAMG

SEQ ID NO: 20 - protein, 10 aa, synthetic construct
  REGION 1..10: note = Nanobody
INISGGNTDY

SEQ ID NO: 21 - protein, 12 aa, synthetic construct
  REGION 1..12: note = Nanobody
AVNRRGQDDYEY

SEQ ID NO: 22 - protein, 130 aa, synthetic construct
  REGION 1..130: note = Nanobody
QVQLQESGGGLVQAGGSLRLSCAASGFTFDDYAMGWFRQAPGKEREFVTAISWSGGNTYYADSVKGRFTISRDNAKNTVYLQMNSLKPEDTAVYYCAAKPRRTYYSGSDYYTSPYEYDYSGQGTQVTVSS

SEQ ID NO: 23 - protein, 123 aa, synthetic construct
  REGION 1..123: note = Nanobody
QVQLQESGGGLVQAGGSLRLSCAASGRTFNSFNMAWFRQAPGKAREFVAGIMWSGTHTRYADSVKGRFTISRDNAKSTVLLQMNSLKPEDTAVYYCAGQIYGDYFKESNMQYWGKGTQVTVSS

SEQ ID NO: 24 - protein, 123 aa, synthetic construct
  REGION 1..123: note = Nanobody
QVQLQESGGGLVQPGGSLRLSCTASGRTFNSFNMAWFRQGPGKAREFVAGIMWSGTHTRYADSVKGRFTISRDNAKSTVLLQMNSLKPEDTAVYYCAGQIYGDYLKESNMQYWGKGTQVTVSS

SEQ ID NO: 25 - protein, 123 aa, synthetic construct
  REGION 1..123: note = Nanobody
QVQLQESGGGLVHTGGSLRLSCAASGRTFSSFNMAWFRQAPGKEREFVAGIMWSGTHTRYADSVKGRATISRDNAKNTVLLQMNSLKPEDTAVYYCAAQIYGDYFKESGMQYWGKGTQVTVSS

SEQ ID NO: 26 - protein, 119 aa, synthetic construct
  REGION 1..119: note = Nanobody
QVQLQESGGGLVQTGGSLRLSCLASGRTFRYYAMGWFRQAPGKEREFVAGINISGSNTDYSDSVKGRFTISKDNAKNMGYLQMNSLKPEDTAVYYCAANRRGPNDYEYWGRGTQVTVSS

SEQ ID NO: 27 - protein, 119 aa, synthetic construct
  REGION 1..119: note = Nanobody
QVQLQESGGGLVQAGDSLRLSCLASGRTFRYYAMGWFRQAPGKEREFVAGINISGSNTDYSDSVKGRFTISKDNAKNMGYLQMNSLKPEDTAVYYCAANRRGRNDYEYWGRGTQVTVSS

SEQ ID NO: 28 - protein, 119 aa, synthetic construct
  REGION 1..119: note = Nanobody
QVQLQESGGGLVQAGGSLRLSCAASGRTFRYYAMGWFRQAPGKEREFVAGINISGGNTDYPDSVKGRFTISRDNAKNTGYLQMNSLKPEDTAVYYCAVNRRGQDDYEYWGRGTQVTVSS

SEQ ID NO: 29 - protein, 5 aa, synthetic construct
  REGION 1..5: note = Linker
GGGGS

SEQ ID NO: 30 - protein, 10 aa, synthetic construct
  REGION 1..10: note = Linker
GGGGSGGGGS

SEQ ID NO: 31 - protein, 15 aa, synthetic construct
  REGION 1..15: note = Linker
GGGGSGGGGSGGGGS

SEQ ID NO: 32 - protein, 20 aa, synthetic construct
  REGION 1..20: note = Linker
GGGGSGGGGSGGGGSGGGGS

SEQ ID NO: 33 - protein, 25 aa, synthetic construct
  REGION 1..25: note = Linker
GGGGSGGGGSGGGGSGGGGSGGGGS

SEQ ID NO: 34 - protein, 30 aa, synthetic construct
  REGION 1..30: note = Linker
GGGGSGGGGSGGGGSGGGGSGGGGSGGGGS

SEQ ID NO: 35 - DNA, 2418 bp, Clostridium difficile
  source 1..2418: mol_type = genomic DNA
atgtctttagttaaccgcaagcaattagaaaagatggctaacgtgcggttccggactcaagaggacgagtacgttgcgatcttagacgcactggaagagtatcataatatgagtgagaatactgtagttgagaagtatctgaagttaaaggacataaactcgttgacagatatatacattgacacttacaaaaaatctggaagaaacaaggcactgaagaagttcaaagagtatttggttacagaggtactggagctgaagaacaataacctgacgccagtcgaaaagaatttgcactttgtgtggattggaggacagattaatgacacggcgataaactacataaatcaatggaaggacgtcaactccgactacaatgtcaatgtcttttatgactccaatgcgtttttaattaataccctgaagaaaacagttgttgaaagtgctataaatgatacacttgagtccttccgtgagaacttgaacgacccccggttcgattataataagttctttcgcaaaagaatggagattatatacgacaaacaaaagaacttcattaattactacaaggcacaacgggaagaaaacccggagttaataatagatgatatagtcaagacctacttgtctaatgaatattccaaggaaatagacgagttaaatacttatattgaggagagtctgaataaaatcactcaaaactcgggaaatgatgtgagaaattttgaggagtttaagaatggggagagctttaatctttacgagcaggagctggtagagcgttggaatcttgctgcggcgtcagacattttgcggatcagcgcccttaaggagatagggggaatgtatttagacgtagacatgttgccagggattcagcccgacctgtttgaatccatcgaaaaaccgtcaagcgtcacggtcgacttttgggagatgaccaaacttgaggctataatgaagtacaaggaatatataccagagtataccagtgagcattttgacatgcttgatgaagaagttcagtcatcatttgagtcagtacttgcaagtaaatctgacaagagtgagatatttagctcgttaggcgatatggaggcttcaccacttgaggtcaagatcgctttcaatagcaaaggaatcatcaaccagggattaatttctgttaaagactcctactgttctaacttgatagtcaaacaaattgaaaaccgttacaagatactgaataatagtcttaacccagccattagcgaggacaacgactttaacacgaccactaacacattcatcgattcaattatggccgaagcaaatgcggacaacggtcggttcatgatggaattagggaagtacctgcgtgtgggcttcttcccggatgtgaagacaaccataaacctgagtggtccggaagcctacgcagcggcatatcaagaccttttgatgtttaaagaaggatctatgaacattcacttaatagaggccgacctgcgtaacttcgaaatctctaaaacgaatatatcgcagtcaacggaacaggagatggcatccctgtggtctttcgacgacgctcgcgcgaaagcacaattcgaggaatacaagcgcaattatttcgaaggtagtctgggggaagatgacaatttagactttagccagaatatcgtggtggacaaagagtatttgttggaaaagatctcaagtttagcccggagctcggagcgcggatatatacactacatagtgcagttacaaggggacaaaatttcatacgaggcggcatgtaatctttttgcaaagacgccctacgatagcgtgctgttccagaaaaacatcgaggactcggagatagcgtattactacaatccaggtgatggggaaatacaggagatagataagtacaagataccatcgattatctccgatcgtcccaagatcaagttgacctttatcggtcacggcaaggatgaattcaacaccgacatttttgcaggttttgacgttgattcgctgtccacagaaattgaggccgcgatcgacctggcgaaggaggatatatcccctaaatctatcgagattaacttacttgggtgtaacatgttctcctactctattaacgtcgaggaaacgtatcccggtaagctgttattaaaagtcaaagacaaaatatccgaactgatgcctagtatcagccaggactcaattatcgtctccgcgaaccagtatgaagttcgtataaactcagagggtcgtcgcgaattattagatcacagcggggaatggattaataaggaggagagcataataaaggacatctcctcgaaagagtatatatcattcaatccgaaggaaaataagataacagttaagagtaagaacttaccagaattgtcaacccttcttcaggagattcgcaac

SEQ ID NO: 36 - DNA, 390 bp, synthetic construct
  misc_feature 1..390: note = Nanobody
  source 1..390: mol_type = other DNA
caggtgcagctgcaggagtctgggggaggcttggtgcaggctggggggtctctgagactctcctgtgcagcctctggattcactttcgatgattatgccatgggctggttccgccaggctccagggaaggagcgtgagtttgtaacagctattagctggagtggtggtaacacatactatgcagactccgtgaagggccgattcaccatctccagagacaacgccaagaacacggtgtatctgcaaatgaacagcctgaaacctgaggacacggccgtttattactgtgcagccaagccccgtcgcacatactatagtggtagtgactactacacgagcccatatgagtatgactactctggccaggggacccaggtcaccgtctcctca

SEQ ID NO: 37 - DNA, 369 bp, synthetic construct
  misc_feature 1..369: note = Nanobody
  source 1..369: mol_type = other DNA
caggtgcagctgcaggagtctgggggaggattggtgcaggctgggggctctctgagactctcctgtgcagcctctggacgcaccttcaatagcttcaacatggcctggttccgccaggctccagggaaggcgcgtgagtttgtagcaggtattatgtggagtggtacgcacacacgctatgcagattccgtgaagggccgattcaccatctccagagacaacgccaagagcacggtgcttctgcaaatgaacagcctgaaacctgaggacacggccgtttattactgtgcaggccaaatctatggcgactatttcaaggagtccaacatgcagtactggggcaaagggacccaggtcaccgtctcctca

SEQ ID NO: 38 - DNA, 369 bp, synthetic construct
  misc_feature 1..369: note = Nanobody
  source 1..369: mol_type = other DNA
caggtgcagctgcaggagtctgggggaggcttggtgcagcctggggggtctctgagactctcctgtacagcctctggacgcaccttcaatagcttcaacatggcctggttccgccagggtccagggaaggcgcgtgagtttgtagcaggtattatgtggagtggtacacacacacgctatgcagattccgtgaagggccgattcaccatctccagagacaacgccaagagcacggtgcttctgcaaatgaacagcctgaaacctgaggacacggccgtttattactgtgcaggccaaatctatggcgactatttgaaggagtccaacatgcaatactggggcaaagggacccaggtcaccgtctcctca

SEQ ID NO: 39 - DNA, 369 bp, synthetic construct
  misc_feature 1..369: note = Nanobody
  source 1..369: mol_type = other DNA
caggtgcagctgcaggagtctgggggaggattggtgcacactgggggctctctgagactcagctgtgcagcctctggacgcaccttcagtagcttcaacatggcctggttccgccaggctccagggaaggagcgtgagtttgtagcaggtattatgtggagtggtacacacacacgctatgcagattccgtgaagggccgcgccaccatctccagagacaacgccaagaacacggtgcttctgcaaatgaacagcctgaaacctgaggacacggccgtttattactgtgcagcccaaatctacggcgactattttaaggagtccggcatgcagtactggggcaaagggacccaggtcaccgtctcctca

SEQ ID NO: 40 - DNA, 357 bp, synthetic construct
  misc_feature 1..357: note = Nanobody
  source 1..357: mol_type = other DNA
caggtgcagctgcaggagtctgggggaggcttggtgcaaactggggggtctctgagactctcctgtttagcctctggacgcaccttccgttactatgccatgggctggttccgccaggctccagggaaggagcgtgagttcgtagcaggtattaacattagtggtagtaacactgactattcagactccgtgaagggccgattcaccatctccaaggacaacgccaagaatatggggtatctgcaaatgaacagcctgaaacctgaggacacggccgtttattactgtgcagcgaaccgtcgcggtcctaatgactatgagtactggggccgggggacccaggtcaccgtctcctca

SEQ ID NO: 41 - DNA, 357 bp, synthetic construct
  misc_feature 1..357: note = Nanobody
  source 1..357: mol_type = other DNA
caggtgcagctgcaggagtctgggggaggattggtgcaggctggggactctctgagactctcctgtttagcctctggacgcaccttccgttactatgccatgggctggttccgccaggctccagggaaggagcgtgagttcgtagcaggtattaatattagtggtagtaacactgactattcagactccgtgaagggccgattcaccatctccaaggacaacgccaagaatatggggtatctgcaaatgaacagcctgaaacctgaggacacggccgtttattactgtgcagcgaaccgtcggggtcgtaatgactatgagtactggggccgggggacccaggtcaccgtctcctca

SEQ ID NO: 42 - DNA, 357 bp, synthetic construct
  misc_feature 1..357: note = Nanobody
  source 1..357: mol_type = other DNA
caggtgcagctgcaggagtctgggggaggattggtgcaggctgggggatctctgagactctcctgtgcagcctctggacgcaccttccgttactatgccatgggctggttccgccaggctccagggaaggagcgtgagttcgtagcaggtattaacattagtggtggtaacactgactatccagactccgtgaagggccgattcaccatctccagagacaacgccaagaacacggggtatctgcaaatgaacagcctgaaacctgaggacacggccgtttattactgtgcagtgaatcgtcgcggtcaagatgattatgagtactggggccgggggacccaggtcaccgtctcctca

SEQ ID NO: 43 - protein, 141 aa, synthetic construct
  REGION 1..141: note = Nanobody
QVQLQESGGGLVQAGGSLRLSCAASGFTFDDYAMGWFRQAPGKEREFVTAISWSGGNTYYADSVKGRFTISRDNAKNTVYLQMNSLKPEDTAVYYCAAKPRRTYYSGSDYYTSPYEYDYSGQGTQVTVSSAAASAHHHHHH

SEQ ID NO: 44 - protein, 134 aa, synthetic construct
  REGION 1..134: note = Nanobody
QVQLQESGGGLVQAGGSLRLSCAASGRTFNSFNMAWFRQAPGKAREFVAGIMWSGTHTRYADSVKGRFTISRDNAKSTVLLQMNSLKPEDTAVYYCAGQIYGDYFKESNMQYWGKGTQVTVSSAAASAHHHHHH

SEQ ID NO: 45 - protein, 134 aa, synthetic construct
  REGION 1..134: note = Nanobody
QVQLQESGGGLVQPGGSLRLSCTASGRTFNSFNMAWFRQGPGKAREFVAGIMWSGTHTRYADSVKGRFTISRDNAKSTVLLQMNSLKPEDTAVYYCAGQIYGDYLKESNMQYWGKGTQVTVSSAAASAHHHHHH

SEQ ID NO: 46 - protein, 134 aa, synthetic construct
  REGION 1..134: note = Nanobody
QVQLQESGGGLVHTGGSLRLSCAASGRTFSSFNMAWFRQAPGKEREFVAGIMWSGTHTRYADSVKGRATISRDNAKNTVLLQMNSLKPEDTAVYYCAAQIYGDYFKESGMQYWGKGTQVTVSSAAASAHHHHHH

SEQ ID NO: 47 - protein, 130 aa, synthetic construct
  REGION 1..130: note = Nanobody
QVQLQESGGGLVQTGGSLRLSCLASGRTFRYYAMGWFRQAPGKEREFVAGINISGSNTDYSDSVKGRFTISKDNAKNMGYLQMNSLKPEDTAVYYCAANRRGPNDYEYWGRGTQVTVSSAAASAHHHHHH

SEQ ID NO: 48 - protein, 130 aa, synthetic construct
  REGION 1..130: note = Nanobody
QVQLQESGGGLVQAGDSLRLSCLASGRTFRYYAMGWFRQAPGKEREFVAGINISGSNTDYSDSVKGRFTISKDNAKNMGYLQMNSLKPEDTAVYYCAANRRGRNDYEYWGRGTQVTVSSAAASAHHHHHH

SEQ ID NO: 49 - protein, 130 aa, synthetic construct
  REGION 1..130: note = Nanobody
QVQLQESGGGLVQAGGSLRLSCAASGRTFRYYAMGWFRQAPGKEREFVAGINISGGNTDYPDSVKGRFTISRDNAKNTGYLQMNSLKPEDTAVYYCAVNRRGQDDYEYWGRGTQVTVSSAAASAHHHHHH

SEQ ID NO: 50 - protein, 135 aa, synthetic construct
  VARIANT 1..135: note = Nanobody
  VARIANT 30..31: note = misc_feature - Xaa can be any naturally occurring amino acid
  VARIANT 52: note = misc_feature - Xaa can be any naturally occurring amino acid
  VARIANT 56: note = misc_feature - Xaa can be any naturally occurring amino acid
  VARIANT 59: note = misc_feature - Xaa can be any naturally occurring amino acid
  VARIANT 99..100: note = misc_feature - Xaa can be any naturally occurring amino acid
  VARIANT 102..103: note = misc_feature - Xaa can be any naturally occurring amino acid
  VARIANT 105..110: note = misc_feature - Xaa can be any naturally occurring amino acid
  VARIANT 112: note = misc_feature - Xaa can be any naturally occurring amino acid
  VARIANT 116: note = misc_feature - Xaa can be any naturally occurring amino acid
QVQLQESGGGLVQAGGSLRLSCAASGRTFXXYAMGWFRQAPGKEREFVAGIXWSGXNTXYADSVKGRFTISRDNAKNTVYLQMNSLKPEDTAVYYCAAXXRXXYXXXXXXYXYWGXGTQVTVSSAAASAHHHHHH

(19) United States
(12) Patent Application Publication
(10) Pub. No.: US 2025/0376506 A1
(43) Pub. Date: Dec. 11, 2025

(54) SINGLE DOMAIN ANTIBODIES FOR PREVENTION OF CLOSTRIDIUM DIFFICILE INFECTION

(71) Applicant: BACTOLIFE A/S, COPENHAGEN Ø (DK)

(72) Inventors: SANDRA WINGAARD THRANE, COPENHAGEN Ø (DK); ANDREAS HOUGAARD LAUSTSEN, COPENHAGEN Ø (DK); MADS AAGE LAUSTSEN, COPENHAGEN Ø (DK); EVERARDO RODRIGUEZ-RODRIGUEZ, COPENHAGEN Ø (DK)

(21) Appl. No.: 18/715,398

(22) PCT Filed: Dec. 2, 2022
(86) PCT No.: PCT/EP2022/084159

§ 371 Date: May 31, 2024

(30) Foreign Application Priority Data
Dec. 2, 2021 (EP) 21211996.0

(51) Int. Cl. C07K 16/12

(52) U.S. Cl. CPC C07K 16/1282; C07K 2317/24; C07K 2317/33; C07K 2317/565; C07K 2317/622; C07K 2317/94

(57) ABSTRACT

Single domain antibodies which bind to Clostridium difficile toxin B and their use in the treatment and prevention of Clostridium difficile infection. Nucleic acids and vectors encoding the single domain antibodies, host cells for expression of the single domain antibodies, methods of manufacture and compositions.

### CROSS-REFERENCE TO RELATED APPLICATIONS

This is a National Stage application of PCT International Application PCT/EP2022/084159, filed on Dec. 2, 2022, which claims the benefit of European Patent Application EP21211996.0 filed on Dec. 2, 2021, both of which are incorporated herein by reference in their entirety.

The material in the xml file, named “ZBM70382-P6146PC00-corrected.xml”, created Mar. 28, 2025, file size of 73,088 bytes, is hereby incorporated by reference.

### TECHNICAL FIELD

The present disclosure relates to the field of nutritional immunology. More particularly, it concerns single domain antibodies directed against Clostridium difficile toxins, particularly Clostridium difficile toxin B.

### BACKGROUND

Clostridium difficile, also known as Clostridioides difficile, is a gram negative bacteria. Clostridium difficile infection (CDI) is the leading cause of antibiotic-associated diarrhoea worldwide. Moreover, CDI is associated with high mortality especially in specific risk groups, such as the elderly, hospitalized patients, as well as immunocompromised individuals. Currently available treatments primarily involve the use of antibiotics as first-line therapy, such as metrodinazole, as well as vancomycin.

Non-antibiotic based therapeutic regimes for the treatment and/or prevention of Clostridium difficile infection are based upon vaccination and passive immunization. Vaccination treatment comprises administering to a patient either a nucleic acid sequence encoding an immunogenic fragment of the Clostridium difficile surface layer protein or a variant or homologue thereof, or an equivalent polypeptide fragment (as disclosed in WO 02/062379). Passive immunotherapy is typically achieved by administering to a patient a monoclonal antibody specific to an immunogen produced by a pathogen. In general, passive immunotherapy is particularly effective in treating immunocompromised patients who are unable to respond to vaccination, and to patients who need immediate therapy and cannot wait for vaccination to take effect. In the case of a Clostridium difficile infection, passive immunization relies on the administration to a patient of toxin-neutralizing polyclonal immune globulin, (as disclosed in WO 99/2030.4), or antibodies raised against the whole bacterium and the toxins (as disclosed in WO 96/07430).

Increasing rates in treatment failure of CDI and recurrence demands for the development of novel treatment and prevention strategies in relation to CDI.

### SUMMARY

In the present disclosure, the inventors provide a novel option for the prevention and/or treatment of Clostridium difficile, by the provision of single domain antibodies (sdAbs) and fusion proteins targeting Clostridium difficile toxin B. The sdAbs disclosed herein display unique properties making them particularly useful for prevention and/or treatment of CDI and the clinical manifestations of same.

The unique properties of the herein disclosed sdAbs make them particularly suitable for oral delivery and for use in food, feed and beverages, e.g. as a dietary supplement which can be used as a gut health/microbiome stabilizer potentially reducing the risk of CDI. The sdAbs disclosed herein preferably have one or more of the following features:
a) prevents and/or reduces Clostridium difficile toxin B cytotoxicity;
b) prevents and/or reduces toxin activity of Clostridium difficile toxin B;
c) prevents and/or reduces one or more Clostridium difficile-mediated symptoms, such as diarrhea, fever, hematochezia, and/or weight loss;
d) reduces risk of Clostridium difficile infection;
e) is stable in the gastrointestinal tract of a subject;
f) is protease stable;
g) is pH stable;
h) is storage stable;
i) is temperature stable; or
j) improves the gut microbiome.

The inventors of the present disclosure have made the surprising discovery of single domain antibodies displaying high binding affinity towards recombinant Clostridium difficile toxin B (TcdB-GT toxin) compared to known sdAbs binding to TcdB-GT toxin, as shown in the examples. Moreover, the inventors found that the single domain antibodies disclosed herein showed high efficacy in blocking of the enzymatic activity of the glycosyltransferase domain (GT) of the TedB toxin compared to these controls.

### DESCRIPTION OF DRAWINGS

FIG. 1. Results of single-domain antibody screening. Fluorescence intensity of different single-domain antibodies against recombinant TcdB-GT toxin. The figure demonstrates how some of the single-domain antibodies screened, showed a higher fluorescence intensity, and thus higher binding efficiency, compared with the single-domain antibody controls E3 and 5D known from Yang et al., 2014.

FIG. 2A. Results of the blocking activity assay. Bars indicate the percentage activity of recombinant TcdB-GT toxin in the absence or presence of each single-domain antibody. Bars with solid color correspond to 0.5 mg of single-domain antibody, and bars with lines correspond to 0.25 mg. In the case where the two single-domain antibody controls were incubated with the TcdB-Toxin (E3-5D), 0.5 mg of each molecule was used. Thus, this figure demonstrates that some of the single domain antibodies had better blocking activity towards the enzymatic activity of the glycosyltransferase domain compared with the single-domain antibody controls E3 and 5D known from Yang et al., 2014.

FIG. 2B. Results of the blocking activity assay and length of CDR3. Bars indicate the percentage activity of recombinant TcdB-GT toxin in the absence or presence of each single-domain antibody. Bars with solid color correspond to 0.5 mg of single-domain antibody, and bars with lines correspond to 0.25 mg. The solid line refers to the length of the CDR3 region of each sdAb. Thus, this figure demonstrates a correlation between the length of the CDR3 region of the single domain antibodies and their blocking activity towards the enzymatic activity of the glycosyltransferase domain compared to the controls.

FIG. 3. Results of aminoacidic sequence analysis. CDRs are highlighted with square brackets. Colors represent physiochemical properties of the amino acids (Zappo classification).

FIG. 4. Results of homology analysis. Cladogram using neighbor joining. The single-domain antibodies were clustered into three families.

FIG. 5. Results of single-domain antibody sequences analysis. The figure shows the length of CDR3 of the different single domain antibodies.

FIG. 6. Blocking activity of native TcdB-GT toxin by selected single domain antibodies. Bars indicate the percentage activity of native TcdB-GT domain in the absence or presence of each single-domain antibody. This figure demonstrates that in the assay tested, the selected single domain antibodies reduce the activity of the native glycosyltransferase domain more efficiently compared with the single-domain antibody control E3 (Yang et al., 2014).

FIG. 7A-B. pH functional stability. A: The bars indicate the percentage binding of selected single domain antibodies against recombinant TcdB-GT after being incubated at four different physiologically relevant pHs. B: The bars indicate the percentage binding of selected single domain antibodies against native TcdB after been incubated at pH 7.4 and 5.5.

The results demonstrate that the selected single domain antibodies are stable and maintain functionality (binding) against their target in a solution that simulated gastrointestinal conditions and is representative for TcdB cell internalization.

FIG. 8A-B. Thermostability. A: Bars indicate the percentage of binding of selected single domain antibodies after incubation for 1 hour at various temperatures. The highest binding value was observed at 25° C., therefore that condition was taken as reference. B: Bars indicate the percentage of binding of single domain antibodies after incubation for 10 sec at high temperatures.

FIG. 9. Shelf stability in liquid. Bars indicate the percentage of binding capacity of single domain antibodies after storage in liquid for 7 days at 4° C. The value obtained from samples stored in PBS 7.4 was taken as a reference for comparison with samples in milk.

FIG. 10. Cross reactivity.

The bars indicate values of absorbance at 450 nm obtained as a result of the binding of the single domains antibodies to different variants of TcdB-GT. The results demonstrate how one example of a single domain antibody according to the present disclosure had a broad cross reactivity to the different TcdB-GT variants. Meanwhile the CD3A bound to 5 variants.

### DETAILED DESCRIPTION

The present disclosure relates to single domain antibodies (sdAbs) and fusion proteins targeting Clostridium difficile toxins, in particular Clostridium difficile toxin B (TcdB-GT toxin).

### Single Domain Antibodies

A nanobody or single domain antibody (sdAb), as used herein, refers to the smallest antigen binding fragment or single variable domain (“VHH”) derived from a naturally occurring heavy chain antibody and is known to the person skilled in the art. Such single domain antibodies can be derived from antibodies raised in Camelidae species, for example in camel, llama, dromedary, alpaca and guanaco. Single domain antibodies may also be synthetically produced, such as by expression in bacteria. Single domain antibodies are antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies.

The term single domain antibody, in its broadest sense, is not limited to a specific biological source or to a specific method of preparation. For example, the single domain antibodies of the disclosure can generally be obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding a such humanized VHH domain; (4) by “camelization” of a naturally occurring VH domain from any animal species, and in particular from a mammalian species, such as from a human being, or by expression of a nucleic acid encoding such a camelized VH domain; (5) by “camelization” of a “domain antibody” or “Dab,” as described in the art, or by expression of a nucleic acid encoding such a camelized VH domain; (6) by using synthetic or semi-synthetic techniques for preparing proteins, polypeptides or other amino acid sequences known per se: (7) by preparing a nucleic acid encoding a single domain antibody using techniques for nucleic acid synthesis known per se, followed by expression of the nucleic acid thus obtained; and/or (8) by any combination of one or more of the foregoing.

### CD3A

In one embodiment, the single domain antibody of the present disclosure is the single domain antibody “CD3A” or is a variant thereof.

In one embodiment, the single domain antibody of the present disclosure is a single domain antibody comprising:
i) a complementary-determining region 1 (CDR1) comprising or consisting of SEQ ID NO: 1 or a variant thereof, wherein one or more amino acids have been altered, with the proviso that no more than 3 amino acids have been so altered, for example wherein 2, or 1 amino acids have been so altered; and
ii) a complementary-determining region 2 (CDR2) comprising or consisting of SEQ ID NO: 2 or a variant thereof, wherein one or more amino acids have been altered, with the proviso that no more than 3 amino acids have been so altered, for example wherein 2, or 1 amino acids have been so altered; and
iii) a complementary-determining region 3 (CDR3) comprising or consisting of SEQ ID NO: 3 or a variant thereof, wherein one or more amino acids have been altered, with the proviso that no more than 3 amino acids have been so altered, for example wherein 2, or 1 amino acids have been so altered.

In one embodiment, no more than 2 amino acids have been altered in each CDR. In one embodiment, no more than 1 amino acid has been altered in each CDR.

In one embodiment, the single domain antibody only has one or more amino acid alterations in CDR3. Thus, in one embodiment, the present disclosure relates to a single domain antibody comprising:
i) a complementary-determining region 1 (CDR1) comprising or consisting of SEQ ID NO: 1; and
ii) a complementary-determining region 2 (CDR2) comprising or consisting of SEQ ID NO: 2; and
iii) a complementary-determining region 3 (CDR3) comprising or consisting of SEQ ID NO: 3 or a variant thereof, wherein one or more amino acids have been altered, with the proviso that no more than 3 amino acids have been so altered, for example wherein 2, or 1 amino acids have been so altered.

In one embodiment, the single domain antibody of the present disclosure is a single domain antibody comprising:
i) a complementary-determining region 1 (CDR1) comprising or consisting of an amino acid sequence according to SEQ ID NO: 1;
ii) a complementary-determining region 2 (CDR2) comprising or consisting of an amino acid sequence according to SEQ ID NO: 2; and
iii) a complementary-determining region 3 (CDR3) comprising or consisting of an amino acid sequence according to SEQ ID NO: 3.

In one embodiment, the single domain antibody comprises or consists of the sequence as set forth in SEQ ID NO: 22, or a sequence having at least 90% sequence identity thereto. In one embodiment, the sequence identity is at least 95%, such as at least 96%, 97%, 98% or 99%. In one embodiment, the sequence variance is outside the CDRs.

In one embodiment the single domain antibody of the present disclosure is a humanised version of the single domain antibody of CD3A as disclosed above.

In some embodiments, the single domain antibody is selected from the group consisting of:
a) a single domain antibody comprising:
   i. a complementary-determining region 1 (CDR1) comprising or consisting of SEQ ID NO: 1; and
   ii. a complementary-determining region 2 (CDR2) comprising or consisting of SEQ ID NO: 2; and
   iii. a complementary-determining region 3 (CDR3) comprising or consisting of SEQ ID NO: 3;
b) a single domain antibody comprising or consisting of the sequence as set forth in SEQ ID NO: 22; and
c) a humanised version of the single domain antibody of a) or b).

### CD1C

In one embodiment, the single domain antibody of the present is disclosure is the single domain antibody “CD1C”, or is a variant thereof. In one embodiment, the single domain antibody of the present disclosure is a single domain antibody comprising:
i) a complementary-determining region 1 (CDR1) comprising or consisting of SEQ ID NO: 4 or a variant thereof, wherein one or more amino acids have been altered, with the proviso that no more than 3 amino acids have been so altered, for example wherein 2, or 1 amino acids have been so altered; and
ii) a complementary-determining region 2 (CDR2) comprising or consisting of SEQ ID NO: 5 or a variant thereof, wherein one or more amino acids have been altered for another amino acid, with the proviso that no more than 3 amino acids have been so altered, for example wherein 2, or 1 amino acids have been so altered; and
iii) a complementary-determining region 3 (CDR3) comprising or consisting of SEQ ID NO: 6 or a variant thereof, wherein one or more amino acids have been altered, with the proviso that no more than 3 amino acids have been so altered, for example wherein 2, or 1 amino acids have been so altered.

In one embodiment, no more than 2 amino acids have been altered in each CDR. In one embodiment, no more than 1 amino acid has been altered in each CDR.

In one embodiment, the single domain antibody only has one or more amino acid alterations in CDR3. Thus, in one embodiment, the present disclosure relates to a single domain antibody comprising:
i) a complementary-determining region 1 (CDR1) comprising or consisting of SEQ ID NO: 4; and
ii) a complementary-determining region 2 (CDR2) comprising or consisting of SEQ ID NO: 5; and
iii) a complementary-determining region 3 (CDR3) comprising or consisting of SEQ ID NO: 6 or a variant thereof, wherein one or more amino acids have been altered, with the proviso that no more than 3 amino acids have been so altered, for example wherein 2, or 1 amino acids have been so altered.

In one embodiment, the single domain antibody of the present disclosure is a single domain antibody comprising:
i) a complementary-determining region 1 (CDR1) comprising or consisting of an amino acid sequence according to SEQ ID NO: 4;
ii) a complementary-determining region 2 (CDR2) comprising or consisting of an amino acid sequence according to SEQ ID NO: 5; and
iii) a complementary-determining region 3 (CDR3) comprising or consisting of an amino acid sequence according to SEQ ID NO: 6.

In one embodiment, the single domain antibody comprises or consists of the sequence as set forth in SEQ ID NO: 23, or a sequence having at least 90% sequence identity thereto. In one embodiment, the sequence identity is at least 95%, such as at least 96%, 97%, 98% or 99%. In one embodiment, the sequence variance is outside the CDRs . . .

In one embodiment the single domain antibody of the present disclosure is a humanised version of the single domain antibody of CD1C as disclosed above.

### CD2A

In one embodiment, the single domain antibody of the present disclosure is the single domain antibody “CD2A” or is a variant thereof. In one embodiment, the single domain antibody of the present disclosure is a single domain antibody comprising:
i) a complementary-determining region 1 (CDR1) comprising or consisting of SEQ ID NO: 7 or a variant thereof, wherein one or more amino acids have been altered, with the proviso that no more than 3 amino acids have been so altered, for example wherein 2, or 1 amino acids have been so altered; and
ii) a complementary-determining region 2 (CDR2) comprising or consisting of SEQ ID NO: 8 or a variant thereof, wherein one or more amino acids have been altered, with the proviso that no more than 3 amino acids have been so altered, for example wherein 2, or 1 amino acids have been so altered; and
iii) a complementary-determining region 3 (CDR3) comprising or consisting of SEQ ID NO: 9 or a variant thereof, wherein one or more amino acids have been altered, with the proviso that no more than 3 amino acids have been so altered, for example wherein 2, or 1 amino acids have been so altered.

In one embodiment, no more than 2 amino acids have been altered in each CDR. In one embodiment, no more than 1 amino acid has been altered in each CDR.

In one embodiment, the single domain antibody only has one or more amino acid alterations in CDR3. Thus, in one embodiment, the present disclosure relates to a single domain antibody comprising:
i) a complementary-determining region 1 (CDR1) comprising or consisting of SEQ ID NO: 7; and
ii) a complementary-determining region 2 (CDR2) comprising or consisting of SEQ ID NO: 8; and
iii) a complementary-determining region 3 (CDR3) comprising or consisting of SEQ ID NO: 9 or a variant thereof, wherein one or more amino acids have been altered, with the proviso that no more than 3 amino acids have been so altered, for example wherein 2, or 1 amino acids have been so altered.

In one embodiment, the single domain antibody of the present disclosure is a single domain antibody comprising:
i) a complementary-determining region 1 (CDR1) comprising or consisting of an amino acid sequence according to SEQ ID NO: 7;
ii) a complementary-determining region 2 (CDR2) comprising or consisting of an amino acid sequence according to SEQ ID NO: 8; and
iii) a complementary-determining region 3 (CDR3) comprising or consisting of an amino acid sequence according to SEQ ID NO: 9.

In one embodiment, the single domain antibody comprises or consists of the sequence as set forth in SEQ ID NO: 24, or a sequence having at least 90% sequence identity thereto. In one embodiment, the sequence identity is at least 95%, such as at least 96%, 97%, 98% or 99%. In one embodiment, the sequence variance is outside the CDRs.

In one embodiment the single domain antibody of the present disclosure is a humanised version of the single domain antibody of CD2A as disclosed above.

### CD6E

In one embodiment, the single domain antibody of the present disclosure is the single domain antibody “CD6E” or is a variant thereof. In one embodiment, the single domain antibody of the present disclosure is a single domain antibody comprising:
i) a complementary-determining region 1 (CDR1) comprising or consisting of SEQ ID NO: 10 or a variant thereof, wherein one or more amino acids have been altered, with the proviso that no more than 3 amino acids have been so altered, for example wherein 2, or 1 amino acids have been so altered; and
ii) a complementary-determining region 2 (CDR2) comprising or consisting of SEQ ID NO: 11 or a variant thereof, wherein one or more amino acids have been altered, with the proviso that no more than 3 amino acids have been so altered, for example wherein 2, or 1 amino acids have been so altered; and
iii) a complementary-determining region 3 (CDR3) comprising or consisting of SEQ ID NO: 12 or a variant thereof, wherein one or more amino acids have been altered, with the proviso that no more than 3 amino acids have been so altered, for example wherein 2, or 1 amino acids have been so altered.

In one embodiment, no more than 2 amino acids have been altered in each CDR. In one embodiment, no more than 1 amino acid has been altered in each CDR.

In one embodiment, the single domain antibody only has one or more amino acid alterations in CDR3. Thus, in one embodiment, the present disclosure relates to a single domain antibody comprising:
i) a complementary-determining region 1 (CDR1) comprising or consisting of SEQ ID NO: 10; and
ii) a complementary-determining region 2 (CDR2) comprising or consisting of SEQ ID NO: 11; and
iii) a complementary-determining region 3 (CDR3) comprising or consisting of SEQ ID NO: 12 or a variant thereof, wherein one or more amino acids have been altered, with the proviso that no more than 3 amino acids have been so altered, for example wherein 2, or 1 amino acids have been so altered.

In one embodiment, the single domain antibody of the present disclosure is a single domain antibody comprising:
i) a complementary-determining region 1 (CDR1) comprising or consisting of an amino acid sequence according to SEQ ID NO: 10;
ii) a complementary-determining region 2 (CDR2) comprising or consisting of an amino acid sequence according to SEQ ID NO: 11; and
iii) a complementary-determining region 3 (CDR3) comprising or consisting of an amino acid sequence according to SEQ ID NO: 12.

In one embodiment, the single domain antibody comprises or consists of the sequence as set forth in SEQ ID NO: 25, or a sequence having at least 90% sequence identity thereto. In one embodiment, the sequence identity is at least 95%, such as at least 96%, 97%, 98% or 99%. In one embodiment, the sequence variance is outside the CDRs.

In one embodiment the single domain antibody of the present disclosure is a humanised version of the single domain antibody of CD6E as disclosed above.

### CD2F

In one embodiment, the single domain antibody of the present disclosure is the single domain antibody “CD2F” or a variant thereof. In one embodiment, the single domain antibody of the present disclosure is a single domain antibody comprising:
i) a complementary-determining region 1 (CDR1) comprising or consisting of SEQ ID NO: 13 or a variant thereof, wherein one or more amino acids have been altered, with the proviso that no more than 3 amino acids have been so altered, for example wherein 2, or 1 amino acids have been so altered; and
ii) a complementary-determining region 2 (CDR2) comprising or consisting of SEQ ID NO: 14 or a variant thereof, wherein one or more amino acids have been altered, with the proviso that no more than 3 amino acids have been so altered, for example wherein 2, or 1 amino acids have been so altered; and
iii) a complementary-determining region 3 (CDR3) comprising or consisting of SEQ ID NO: 15 or a variant thereof, wherein one or more amino acids have been altered, with the proviso that no more than 3 amino acids have been so altered, for example wherein 2, or 1 amino acids have been so altered.

In one embodiment, no more than 2 amino acids have been altered in each CDR. In one embodiment, no more than 1 amino acid has been altered in each CDR.

In one embodiment, the single domain antibody only has one or more amino acid alterations in CDR3. Thus, in one embodiment, the present disclosure relates to a single domain antibody comprising:
i) a complementary-determining region 1 (CDR1) comprising or consisting of SEQ ID NO: 13; and
ii) a complementary-determining region 2 (CDR2) comprising or consisting of SEQ ID NO: 14; and
iii) a complementary-determining region 3 (CDR3) comprising or consisting of SEQ ID NO: 15 or a variant thereof, wherein one or more amino acids have been altered, with the proviso that no more than 3 amino acids have been so altered, for example wherein 2, or 1 amino acids have been so altered.

In one embodiment, the single domain antibody of the present disclosure is a single domain antibody comprising:
i) a complementary-determining region 1 (CDR1) comprising or consisting of an amino acid sequence according to SEQ ID NO: 13;
ii) a complementary-determining region 2 (CDR2) comprising or consisting of an amino acid sequence according to SEQ ID NO: 14; and
iii) a complementary-determining region 3 (CDR3) comprising or consisting of an amino acid sequence according to SEQ ID NO: 15.

In one embodiment, the single domain antibody comprises or consists of the sequence as set forth in SEQ ID NO: 26, or a sequence having at least 90% sequence identity thereto. In one embodiment, the sequence identity is at least 95%, such as at least 96%, 97%, 98% or 99%. In one embodiment, the sequence variance is outside the CDRs.

In one embodiment the single domain antibody of the present disclosure is a humanised version of the single domain antibody of CD2F as disclosed above.

### CD2C

In one embodiment, the single domain antibody of the present disclosure is the single domain antibody “CD2C” or a variant thereof. In one embodiment, the single domain antibody of the present disclosure is a single domain antibody comprising:
i) a complementary-determining region 1 (CDR1) comprising or consisting of SEQ ID NO: 16 or a variant thereof, wherein one or more amino acids have been altered, with the proviso that no more than 3 amino acids have been so altered, for example wherein 2, or 1 amino acids have been so altered; and
ii) a complementary-determining region 2 (CDR2) comprising or consisting of SEQ ID NO: 17 or a variant thereof, wherein one or more amino acids have been altered, with the proviso that no more than 3 amino acids have been so altered, for example wherein 2, or 1 amino acids have been so altered; and
iii) a complementary-determining region 3 (CDR3) comprising or consisting of SEQ ID NO: 18 or a variant thereof, wherein one or more amino acids have been altered, with the proviso that no more than 3 amino acids have been so altered, for example wherein 2, or 1 amino acids have been so

In one embodiment, no more than 2 amino acids have been altered in each CDR. In one embodiment, no more than 1 amino acid has been altered in each CDR.

In one embodiment, the single domain antibody only has one or more amino acid alterations in CDR3. Thus, in one embodiment, the present disclosure relates to a single domain antibody comprising:
i) a complementary-determining region 1 (CDR1) comprising or consisting of SEQ ID NO: 16; and
ii) a complementary-determining region 2 (CDR2) comprising or consisting of SEQ ID NO: 17; and
iii) a complementary-determining region 3 (CDR3) comprising or consisting of SEQ ID NO: 18 or a variant thereof, wherein one or more amino acids have been altered, with the proviso that no more than 3 amino acids have been so altered, for example wherein 2, or 1 amino acids have been so altered.

In one embodiment, the single domain antibody of the present disclosure is a single domain antibody comprising:
i) a complementary-determining region 1 (CDR1) comprising or consisting of an amino acid sequence according to SEQ ID NO: 16;
ii) a complementary-determining region 2 (CDR2) comprising or consisting of an amino acid sequence according to SEQ ID NO: 17; and
iii) a complementary-determining region 3 (CDR3) comprising or consisting of an amino acid sequence according to SEQ ID NO: 18.

In one embodiment, the single domain antibody comprises or consists of the sequence as set forth in SEQ ID NO: 27, or a sequence having at least 90% sequence identity thereto. In one embodiment, the sequence identity is at least 95%, such as at least 96%, 97%, 98% or 99%. In one embodiment, the sequence variance is outside the CDRs.

In one embodiment the single domain antibody of the present disclosure is a humanised version of the single domain antibody of CD2C as disclosed above.

### CD1B

In one embodiment, the single domain antibody of the present disclosure is the single domain antibody “CD1B” or a variant thereof. In one embodiment, the single domain antibody of the present disclosure is a single domain antibody comprising:
i) a complementary-determining region 1 (CDR1) comprising or consisting of SEQ ID NO: 19 or a variant thereof, wherein one or more amino acids have been altered, with the proviso that no more than 3 amino acids have been so altered, for example wherein 2, or 1 amino acids have been so altered; and
ii) a complementary-determining region 2 (CDR2) comprising or consisting of SEQ ID NO: 20 or a variant thereof, wherein one or more amino acids have been altered, with the proviso that no more than 3 amino acids have been so altered, for example wherein 2, or 1 amino acids have been so altered; and
iii) a complementary-determining region 3 (CDR3) comprising or consisting of SEQ ID NO: 21 or a variant thereof, wherein one or more amino acids have been altered, with the proviso that no more than 3 amino acids have been so altered, for example wherein 2, or 1 amino acids have been so altered.

In one embodiment, no more than 2 amino acids have been altered in each CDR. In one embodiment, no more than 1 amino acid has been altered in each CDR.

In one embodiment, the sdAb only has one or more amino acid alterations in CDR3. Thus, in one embodiment, the present disclosure relates to a single domain antibody comprising:
i) a complementary-determining region 1 (CDR1) comprising or consisting of SEQ ID NO: 19; and
ii) a complementary-determining region 2 (CDR2) comprising or consisting of SEQ ID NO: 20; and
iii) a complementary-determining region 3 (CDR3) comprising or consisting of SEQ ID NO: 21 or a variant thereof, wherein one or more amino acids have been altered, with the proviso that no more than 3 amino acids have been so altered, for example wherein 2, or 1 amino acids have been so

In one embodiment, the single domain antibody of the present disclosure is a single domain antibody comprising:
i) a complementary-determining region 1 (CDR1) comprising or consisting of an amino acid sequence according to SEQ ID NO: 19;
ii) a complementary-determining region 2 (CDR2) comprising or consisting of an amino acid sequence according to SEQ ID NO: 20; and
iii) a complementary-determining region 3 (CDR3) comprising or consisting of an amino acid sequence according to SEQ ID NO: 21.

In one embodiment, the single domain antibody comprises or consists of the sequence as set forth in SEQ ID NO: 28, or a sequence having at least 90% sequence identity thereto. In one embodiment, the sequence identity is at least 95%, such as at least 96%, 97%, 98% or 99%. In one embodiment, the sequence variance is outside the CDRs.

In one embodiment the single domain antibody of the present disclosure is a humanised version of the single domain antibody of CD1B as disclosed above.

### Sequence Modifications

In one embodiment the present disclosure allows for minor variations in the CDRs since such CDR variants can retain the activity of and in some cases even improve the activity of the sdAb. The data of the inventors indicate that it may be beneficial to increase the degree of aromatic and/or positively charged amino acids in the CDR(s) of the single domain antibody, particularly in CDR3. Thus, in some embodiments the CDR sequence of any sdAb disclosed herein may be altered, with the proviso that no more than 3 amino acids have been so altered, for example wherein 2, or 1 amino acids have been so altered in each CDR.

In one embodiment the alteration of one or more amino acids comprises a substitution, a deletion or an insertion.

In one embodiment the alteration of one or more amino acids comprises or is a substitution.

In one embodiment, the alteration comprises or is a deletion.

In one embodiment, the alteration comprises or is an insertion.

In one embodiment, the alteration enhances the degree of aromatic and/or positively charged amino acids in the CDR(s) of the single domain antibody. In some embodiments, the degree of aromatic and/or positively charged amino acids in the CDR(s) of the single domain antibody is increased by the alteration. In some embodiments, the degree of aromatic and/or positively charged amino acids in the CDR(s) of the single domain antibody is decreased by the alteration.

For example, the alteration may comprise a substitution and/or an insertion of one or more aromatic amino acids, wherein the aromatic amino acid is an α-amino acid comprising an aromatic group, including aromatic hydrocarbon and aromatic heterocyclic groups in the side-chain thereof.

In one embodiment, the aromatic amino acid is phenylalanine, tryptophan, tyrosine or histidine.

Alternatively or additionally, the alteration may comprise a substitution and/or an insertion of one or more positively charged amino acids, wherein the positively charged amino acid is an amino acid in which the R groups have a net positive charge at pH 7.0.

In one embodiment, the positively charged amino acid is lysine, arginine or histidine.

In one embodiment the alteration of one or more amino acids is in CDR3.

In some embodiments, the alteration is a substitution of an alanine with a glycine.

### Single Domain Antibodies Features

The sdAbs of the present disclosure are useful in ameliorating and/or preventing CDI, reducing the risk of CDI, as well as in the treatment of CDI. To be useful for oral dosage, the sdAbs disclosed herein should be capable of blocking the activity of TcdB following passage through the GI tract. This means that the sdAbs should remain stable and retain activity after passage through the GI tract, be capable of binding TcdB in its native form in the gut lumen under neutral pH, have a high binding affinity to efficiently associate with toxin in a complex environment (e.g. have a Kd<10 nM), remain bound during, or block initiation of endocytosis, and be capable of blocking the cytotoxic effect of TcdB-GT, i.e. efficiently inhibit the glycosyltranferase activity of TcdB-GT.

In one embodiment the sbAbs disclosed herein efficiently bind to their target TcdB and have a Kd<10 nM.

The sdAbs disclosed herein preferably have one or more of the following features:
a) prevents and/or reduces Clostridium difficile toxin B cytotoxicity;
b) prevents and/or reduces toxin activity of Clostridium difficile toxin B;
c) prevents and/or reduces one or more Clostridium difficile-mediated symptoms, such as diarrhea, fever, hematochezia, and/or weight loss;
d) reduces risk of Clostridium difficile infection;
e) is stable in the gastrointestinal tract of a subject;
f) is protease stable;
g) is pH stable;
h) is storage stable;
i) is temperature stable; and/or
j) improves the gut microbiome.

In one embodiment the single domain antibody prevents and/or reduces Clostridium difficile toxin B cytotoxicity.

In one embodiment the single domain antibody prevents and/or reduces toxin activity of Clostridium difficile toxin B.

In one embodiment, the single domain antibody prevents and/or reduces one or more Clostridium difficile-mediated symptoms, such as diarrhea, fever, hematochezia, and/or weight loss.

In another embodiment the provided single domain antibody reduces risk of Clostridium difficile infection.

Clostridium difficile causes a spectrum of diseases ranging from mild diarrhoea to fulminant pseudomembranous colitis (PMC), which are collectively referred to as Clostridium difficile antibiotic-associated diarrhoea (CDAD). CDAD is a common, iatrogenic, nosocomial disease associated with substantial morbidity and mortality, especially in the elderly. Mainly two factors are known to play an important role in the pathogenesis of CDAD, which entails the suppression of the resident intestinal flora by the administration of antibiotics, and the production of two high molecular weight toxins by Clostridium difficile, namely exotoxin A and exotoxin B. Exotoxins A and B are cytotoxic, enterotoxic and proinflammatory.

In one embodiment the single domain antibody prevents and/or reduces Clostridium difficile antibiotic-associated diarrhoea (CDAD).

In another embodiment, the single domain antibody disclosed herein improves the gut microbiome.

In another embodiment, the single domain antibody blocks the enzymatic activity of the glycosyltransferase domain of Clostridium difficile toxin B. In one embodiment, the glycosyltransferase domain has an amino acid sequence according to SEQ ID NO: 35.

In some embodiments, the single domain antibody is capable of blocking at least 20%, such as at least 30%, such as at least 40%, such as at least 50%, such as at least 60%, such as at least 70%, such as at least 80% of the enzymatic activity of the glycosyltransferase domain of Clostridium difficile toxin B.

In another embodiment, the single domain antibody inhibits the biological activity of Clostridium difficile toxin B (TcdB) in stimulating cell invasion.

The single domain antibodies provided herein are preferably stable in the gastrointestinal tract of a subject. Thus, they are usually both protease stable and pH stable and can withstand the harsh conditions of the gastrointestinal tract while retaining their biological activity.

By “stable” herein is meant that at least 50% of the original binding activity is retained. More preferably at least 60%, 70% or 80% of the original binding activity is retained.

In one embodiment at least 60% of the original binding activity is retained.

In one embodiment at least 70% of the original binding activity is retained.

In one embodiment at least 80% of the original binding activity is retained.

In one embodiment at least 90% of the original binding activity is retained.

In some embodiments, at least 50%, such as at least 60%, such as at least 70%, such as at least 80% of the original binding activity against TcdB is retained at pH 5.5.

To determine whether a sdAb is stable in the gastrointestinal tract, one can measure the stability in simulated gastric fluid (SGF) and/or simulated intestinal fluid (SIF).

The term “simulated gastric fluid” or “SGF” used herein refers to an aqueous solution utilized in dissolution testing to mimic the conditions of the stomach. On the other hand the term “simulated intestinal fluid” or “SIF” used herein refers to an aqueous solution utilized in dissolution testing to mimic the conditions of the intestines.

In one embodiment, the single domain antibody is stable in SGF e.g. in a solution comprising 0.05-0.15 M NaCl, such as 0.05, 0.06, 0.07, 0.08, 0.09, 0.10, 0.11, 0.12, 0.13, 0.14 or 0.15 M NaCl and 0.02-0.013 M HCl, such as 0.02, 0.03, 0.04, 0.05, 0.06, 0.07, 0.08, 0.09, 0.10, 0.12, or 0.013 M, PH ˜1.5 for at least 0.5 hours, such as at least for 1 hour.

In one embodiment, the single domain antibody is stable in SIF, e.g. in a solution comprising 0.011-0.021 M NaOH, such as 0.011, 0.012, 0.013, 0.014, 0.015, 0.016, 0.017, 0.018, 0.019, 0.020, or 0.021 M NaOH and 0.027-0.036 M K₃PO₄, such as 0.027, 0.028, 0.029, 0.030, 0.031, 0.032, 0.033, 0.034, 0.035, 0.036 M K₃PO₄, PH ˜7 for at least 0.5 hours, such as at least for 1 hour.

In one embodiment the single domain antibody is protease stable. In one embodiment, the single domain is stable in 50-550 U/ml pepsin, such as 50, 100, 220, 500, or 550 U/ml pepsin in SGF and 12-150 U/ml of pancreatin, such as 12, 25, 50, 100, and 150 U/ml of pancreatin in SIF for at least 0.5 hours, such as at least for 1 hour.

In one embodiment the single domain antibody is pH stable. In one embodiment, the single domain antibody is stable at a pH of 1.5 for at least 0.5 hours, such as at least for 1 hour.

In one embodiment the single domain antibody is stable at a pH of 4 for at least 0.5 hours, such as at least for 1 hour.

In another embodiment the single domain antibody is storage stable. By storage stable is meant that the sdAb can be stored for extended periods of time under various conditions as explained further herein below while retaining at least 50% of the original binding activity. More preferably at least 60%, 70% or 80% of the original binding activity is retained.

In a particular embodiment, the single domain antibody is stable at negative degrees, for example between −15 and −25° C., such as being stable at −15° C., −16° C., −17° C., −18° C., −19° C., −20° C., −21° C., −22° C., −23° C., −24° C. and −25° C., such as for at least 10 days, more preferably at least 15 days, 20 days, 25 days, 30 days, 60 days or more.

In another embodiment, the single domain antibody is stable at positive degrees, for example between 0° C. and 4° C., such as at 0° C., 1° C., 2° C., 3° C., and 4° C. and at room temperatures, such as between 20° C. and 30° C., such as at 20° C., 21° C., 22° C., 23° C., 24° C., 25° C., 26° C., 27° C., 28° C., 29° C. and 30° C. for at least 10 days, more preferably at least 15 days, 20 days, 25 days, 30 days or more.

In one embodiment, the single domain antibody is stored and is stable in saline phosphate solution or in dry form.

In one embodiment the single domain antibody is stable at elevated temperatures, such as at 25° C., 30° C., 37° C., 45° C., 50° C., 60° C., 75° C. and 80° C. for 1 h and at 85° C., 86° C., 87° C., 88° C., 89° C., 90° C., 91° C., 92° C., 93° C., 95° C. for at least 5 seconds, such as 10 or 15 seconds. Thus, the sdAb is able to withstand harsh conditions that may be involved in food, feed and beverage processing, such as able to tolerate pasteurization, which may be performed at 72° C. for 15 seconds.

In one embodiment, the sdAb of the present disclosure is stable in dry form, e.g. as a dry powder, such as in freeze-dried form for at least 4 months at room temperature (25° C.), such as for at least 5 months or 6 months.

In one embodiment, the sdAb of the present disclosure is stable as a solid with medium water activity for 2 months at room temperature (25° C.).

In one embodiment, the sdAb of the present disclosure is stable in a liquid with 3<pH<7 for at least 4 days, more preferably for at least 5, 6 or 7 days at 4° C.

In one embodiment, the sdAb of the present disclosure is stable in different food products or beverages such as in a low moisture food matrix (LMF; water activity <0.6), an intermediate moisture food matrix (IMF; 0.6<a_{w}<0.85) and/or in a high moisture food matrix (HMF; a_{w}>0.85) as shown in the below table so that the sdAb of the present disclosure can be used in such foods or beverages as a dietary supplement/food ingredient and can retain sufficient activity, i.e. it is stable, throughout the normal shelf-life of such products.

| | Water | | Stability |
|---|---|---|---|
| Food matrix | activity | Typical products | examples |
| Low | a_{w} < 0.6 | Powdered (sports | (Fermented) milk |
| moisture | | nutrition, infant | protein |
| (LMF) | | formula, dietary | hydrolysates, 6 |
| | | supplements) | months at RT |
| | | | Egg protein |
| | | | hydrolysate, 2 |
| | | | months at RT |
| Intermediate | 0.6 < a_{w} < 0.85 | High nutrition | Whey protein bars, |
| moisture (IMF) | | protein bars | 26 days at RT |
| High moisture | a_{w} > 0.85 | Protein beverages | Milk, 7 days at 4 C. |
| (HMF) | | | |

### Fusion Proteins

The present disclosure also provides fusion proteins comprising at least one of the single domain antibodies as described herein. Such fusion protein can be assembled by methods known to the person skilled in the art. Preferably, the fusion protein is recombinantly designed by fusing gene sequences in vitro. In some embodiments, the fusion protein further comprises a linker connecting the sbAbs.

In one embodiment, a fusion protein comprising a single domain antibody as described herein and one or more further single domain antibodies, and optionally one or more linkers is provided.

In one embodiment, the fusion protein is a homodimer, i.e. containing two identical sdAbs, or a heterodimer, i.e. containing two different sdAbs.

In one embodiment, the one or more further single domain antibodies bind to Clostridium difficile toxin B and/or Clostridium difficile toxin A.

In a preferred embodiment, the one or more further single domain antibodies are single domain antibodies as provided in the present disclosure.

In one embodiment, the fusion protein comprises any combination of the sdAbs as disclosed herein (including variants thereof) according to the below table.

| | CD3A | CD1C | CD2A | CD6E | CD2F | CD2C | CD1B |
|---|---|---|---|---|---|---|---|
| CD3A | X | | | | | | |
| CD1C | X | X | | | | | |
| CD2A | X | X | X | | | | |
| CD6E | X | X | X | X | | | |
| CD2F | X | X | X | X | X | | |
| CD2C | X | X | X | X | X | X | |
| CD1B | X | X | X | X | X | X | X |

Thus, in one embodiment, the fusion protein comprises the single domain antibody CD3A and at least one of CD3A, CD1C, CD2A, CD6E, CD2F, CD2C or CD1B.

In a particular embodiment, the fusion protein is a homodimer of CDR3A as disclosed herein.

In one embodiment, the fusion protein comprises the single domain antibody CD1C and at least one of CD3A, CD1C, CD2A, CD6E, CD2F, CD2C or CD1B.

In a particular embodiment, the fusion protein is a homodimer of CD1C as disclosed herein.

In one embodiment, the fusion protein comprises the single domain antibody CD2A and at least one of CD3A, CD1C, CD2A, CD6E, CD2F, CD2C or CD1B.

In a particular embodiment, the fusion protein is a homodimer of CD2A as disclosed herein.

In one embodiment, the fusion protein comprises the single domain antibody CD6E and at least one of CD3A, CD1C, CD2A, CD6E, CD2F, CD2C or CD1B.

In a particular embodiment, the fusion protein is a homodimer of CD6E as disclosed herein.

In one embodiment, the fusion protein comprises the single domain antibody CD2F and at least one of CD3A, CD1C, CD2A, CD6E, CD2F, CD2C or CD1B.

In a particular embodiment, the fusion protein is a homodimer of CD2F as disclosed herein.

In one embodiment, the fusion protein comprises the single domain antibody CD2C and at least one of CD3A, CD1C, CD2A, CD6E, CD2F, CD2C or CD1B.

In a particular embodiment, the fusion protein is a homodimer of CD2C as disclosed herein.

In one embodiment, the fusion protein comprises the single domain antibody CD1B and at least one of CD3A, CD1C, CD2A, CD6E, CD2F, CD2C or CD1B.

In a particular embodiment, the fusion protein is a homodimer of CD1B as disclosed herein.

The fusion protein of the present disclosure is usually at least as stable and active as described herein in relation to the individual sdAbs of the present disclosure.

In a preferred embodiment the fusion protein comprises a linker connecting the sdAbs.

In one embodiment the linker is a GS linker, i.e. a linker which comprises or consists of glycine and serine residues. Such linkers are well known in the art.

In one embodiment the linker is a GS linker of the structure (GₓS)ₙ, where x may be a number between 1 to 10, preferably 2 to 5, and n refers to a number of repeats of the GxS sequence, where n may be between 1 to 10, preferably 2 to 5.

Examples of suitable linkers according to the present disclosure include a GGGGS linker (SEQ ID NO: 29), a GGGGSGGGGS linker (SEQ ID NO: 30), a GGGGSGGGGSGGGGS linker (SEQ ID NO: 31), a GGGGSGGGGSGGGGSGGGGS linker (SEQ ID NO: 32), a GGGGSGGGGSGGGGSGGGGSGGGGS linker (SEQ ID NO: 33), or a GGGGSGGGGGGGGSGGGGSGGGGSGGGGS linker (SEQ ID NO: 34).

### Nucleic Acids and Vectors

The present disclosure also provides isolated nucleic acids and vectors encoding the sbAbs and fusion proteins disclosed herein.

In one embodiment, the isolated nucleic acid molecule comprises or consists of SEQ ID NO: 36, SEQ ID NO: 37, SEQ ID NO: 38, SEQ ID NO: 39, SEQ ID NO: 40, SEQ ID NO: 41 and/or SEQ ID NO: 42.

In one embodiment, the provided isolated nucleic acid is codon-optimized for a host cell wherein said nucleic acid molecule is expressed.

In one embodiment, an expression vector comprising the nucleic acid molecule encoding the sbAbs and fusion proteins disclosed herein is provided. The expression vector may be any vector suitable for expression of the herein disclosed nucleic acids.

In one embodiment, the vector is a viral vector, such as an adenoviral vector.

The vector may comprise a promoter to enhance expression of the nucleic acid molecule in at least some host cells.

In one embodiment, the nucleic acid molecule is operably linked to one or more control sequences, such as an inducible promoter, to direct its expression.

### Host Cells and Methods of Manufacture

In one aspect, the present disclosure relates to a recombinant host cell, which is a cultured cell that has been transformed or transfected with a polypeptide-encoding nucleic acid, which can then be expressed in the host cell.

The phrase “recombinant host cell” can be used to denote a host cell that has been transformed or transfected with a nucleic acid to be expressed. Constructs comprising the sequences of interest may be introduced into a host cell by standard techniques. These techniques include transfection, infection, bolistic impact, electroporation, microinjection, scraping, or any other method which introduces the sequences of interest into the host cell as known to a person of skill. A host cell which has been manipulated by any method to take up a DNA sequence, construct or vector will be referred to as “transformed” or “recombinant” herein.

In one embodiment, the present disclosure provides a recombinant host cell comprising the nucleic acid molecule or the expression vector as disclosed herein.

In one embodiment the host cell is a bacterium, a plant, a fungus, such as a yeast, or a mammalian cell.

In one embodiment, the host cell is a bacterium, such as a bacillus, such as a Bacillus licheniformis, Bacillus subtilis, or Bacillus lactobacillus, a Lactobacillus spp., or a Bifidobacterium spp.

In one embodiment, the host cell is a yeast, such as a yeast selected from the genus of pichia, komagataella, hansenula and saccharomyces.

In one embodiment, the host cell is an Aspergillus fungus. In one embodiment, the host cell is a fungus selected from Aspergillus oryzae and Aspergillus niger.

In one embodiment, the present disclosure relates to a method of producing the provided single domain antibody or the fusion protein, the method comprising culturing the host cell as disclosed herein under conditions wherein the single domain antibody or fusion protein is expressed. The method may further comprise a step of purifying and/or isolating the single domain antibody molecule or fusion protein.

### Compositions and Pharmaceutical Uses

The present disclosure further provides a composition comprising a single domain antibody, a fusion protein, a nucleic acid, a vector, and/or a host cell as disclosed herein, optionally further comprising one or more excipients. The composition may further comprise one more buffers, diluents, carriers and/or adjuvants adjusted to the intended use of the composition.

For pharmaceutical compositions, all components of the composition should be pharmaceutically acceptable. By “pharmaceutically acceptable” we mean a non-toxic material that does not decrease the effectiveness of the sbAb. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000)).

The composition may further comprise one or more further active components such as an antibiotics, fecal matter transfer, monoclonal antibodies, probiotics and/or prebiotics.

In one embodiment the composition is a pharmaceutical composition.

In one embodiment, the present disclosure provides a single domain antibody, a fusion protein, a nucleic acid, a vector, a host cell or a pharmaceutical composition as described herein for use as a medicament.

In one embodiment, the present disclosure provides a single domain antibody, a fusion protein, a nucleic acid, a vector, a host cell or a pharmaceutical composition as disclosed herein for use in the prevention or treatment of Clostridium difficile infection in a subject. Treatment encompasses both curative and ameliorative treatment. By ameliorative treatment is meant a treatment that results in the improvement of one or more symptoms of Clostridium difficile infection in a subject.

In one embodiment the subject is a human, e.g. an elderly subject, such as a subject of more than 65 years of age, and/or an immunocompromised subject.

In one embodiment the present disclosure provides a method for prevention and/or treatment of Clostridium difficile infection in a subject in need thereof, said method comprising administering to the subject a single domain antibody, a fusion protein, a nucleic acid, a vector, a host cell or a composition as disclosed herein.

In one embodiment the present disclosure provides a method for reducing the risk of Clostridium difficile infection in a subject in need thereof, said method comprising administering to the subject a single domain antibody, a fusion protein, a nucleic acid, a vector, a host cell or a composition as disclosed herein.

The single domain antibody, fusion protein, nucleic acid, vector, host cell or composition as disclosed herein may be administered in any manner deemed suitable by a person of skill. Enteral administration is particularly preferred, such as orally as a food supplement, as a tablet or a gel, or via gastric intubation.

### Dietary Compositions and Dietary Uses

In one embodiment, the present disclosure provides a dietary/nutraceutical composition comprising a single domain antibody, a fusion protein, a nucleic acid, a vector, a host cell or a composition as disclosed herein. Such dietary composition are considered useful for improving the gut microbiome and for reducing the risk of Clostridium difficile infection.

In one embodiment, the dietary composition comprises one or more of prebiotics, probiotics, synbiotics, proteins, lipids, carbohydrates, vitamins, fibers, and/or nutrients, such as dietary minerals.

In a further embodiment, the disclosure relates to the use of a single domain antibody, a fusion protein, a nucleic acid, a vector, and/or a host cell as disclosed herein as a food ingredient or dietary supplement. In some embodiments, said use is non-medical.

In a further embodiment, the disclosure relates to the use of a single domain antibody, a fusion protein, a nucleic acid, a vector, and/or a host cell as disclosed herein as a food or beverage additive. In some embodiments, said use is non-medical.

In a further embodiment, the disclosure relates to the use of a single domain antibody, a fusion protein, a nucleic acid, a vector, and/or a host cell as disclosed herein as a food or beverage preservative. In some embodiments, said use is non-medical.

### Detections Methods

The single domain antibodies provided herein may also find use in a detection method for detecting Clostridium difficile in a sample.

Thus, in one aspect, the present disclosure provides a method for detecting Clostridium difficile, wherein the method comprises the steps of:
a) providing a sample;
b) contacting the sample with one of more single domain antibodies of the present disclosure; and
c) detecting the complex between the sample and the one or more single domain antibodies of the present disclosure.

In one embodiment, the sample is an isolated sample. Said isolated sample may be a sample isolated from a non-animally derived sample, such as from an earth sample, a foodstuff sample or a water sample. In some embodiments, said sample is a sample not isolated from an animal.

Step b) of the method may further comprises a step of washing the sample, thereby removing any unbound antibody.

In a further embodiment, the method comprises detecting the complex of step c) e.g. by western blotting, ELISA, LFA, microscopy, flow cytometry; TRF, and/or by immunocytochemistry.

In one embodiment, the single domain antibody comprises a detection label, such as a colorimetric, a fluorescent, a luminescent, a magnetic, or a paramagnetic label, or is biotinylated.

The detection method is usually an in vitro method and is performed on samples isolated from a subject.

### Items 1

1. A single domain antibody which binds to Clostridium difficile toxin B, wherein the single domain antibody has one or more of the following features:
   a) prevents and/or reduces Clostridium difficile toxin B cytotoxicity;
   b) prevents and/or reduces toxin activity of Clostridium difficile toxin B;
   c) prevents and/or reduces one or more Clostridium difficile-mediated symptoms, such as diarrhea, fever, hematochezia, and/or weight loss;
   d) reduces risk of Clostridium difficile infection;
   e) is stable in the gastrointestinal tract of a subject;
   f) is protease stable;
   g) is pH stable;
   h) is storage stable;
   i) is temperature stable; and/or
   j) improves the gut microbiome.
2. The single domain antibody according to item 1, wherein the single domain antibody is selected from the group consisting of:
   a) a single domain antibody comprising:
      i. a complementary-determining region 1 (CDR1) comprising or consisting of SEQ ID NO: 1 or a variant thereof, wherein one or more amino acids have been altered, with the proviso that no more than 3 amino acids have been so altered, for example wherein 2, or 1 amino acids have been so altered; and
      ii. a complementary-determining region 2 (CDR2) comprising or consisting of SEQ ID NO: 2 or a variant thereof, wherein one or more amino acids have been altered, with the proviso that no more than 3 amino acids have been so altered, for example wherein 2, or 1 amino acids have been so altered; and
      iii. a complementary-determining region 3 (CDR3) comprising or consisting of SEQ ID NO: 3 or a variant thereof, wherein one or more amino acids have been altered, with the proviso that no more than 3 amino acids have been so altered, for example wherein 2, or 1 amino acids have been so altered;
   b) a single domain antibody comprising or consisting of the sequence as set forth in SEQ ID NO: 22, or a sequence having at least 90% sequence identity thereto; and
   c) a humanised version of the single domain antibody of a) or b).
3. The single domain antibody according to any one of the preceding items, wherein any sequence variance is outside the complementary-determining regions (CDRs).
4. A fusion protein comprising a single domain antibody as defined in any one of the preceding items and one or more further single domain antibodies, and optionally one or more linkers, such as wherein the linker is a GS linker.
5. The fusion protein according to item 4, wherein the fusion protein is a homodimer.
6. An isolated nucleic acid molecule encoding the single domain antibody according to any one of items 1-3 or the fusion protein according to any one of items 4-5.
7. An expression vector comprising the nucleic acid molecule according to item 6.
8. A recombinant host cell comprising the nucleic acid molecule according to item 6 or the expression vector according to item 7.
9. A method of producing the single domain antibody according to any one of items 1-3 or the fusion protein according to any one of items 4-5, the method comprising culturing the host cell according to item 8 under conditions wherein the single domain antibody or fusion protein is expressed.
10. A composition comprising the single domain antibody according to any one of items 1-3, the fusion protein according to any one of items 4-5, the nucleic acid according to item 6, the vector according to item 7, and/or the host cell according to item 8, optionally further comprising one or more excipients.
11. A single domain antibody according to any one of items 1-3, the fusion protein according to any one of items 4-5, the nucleic acid according to item 6, the vector according to item 7, the host cell according to item 8, and/or the composition according to item 10 for use in the prevention or treatment of Clostridium difficile infection in a subject.
12. The single domain antibody, the fusion protein, the nucleic acid, the vector, the host cell or the composition for use according to item 11, wherein the single domain antibody, the fusion protein, the nucleic acid, the vector, the host cell or the composition is administered enterally, such as orally, such as a food supplement, as a tablet or a gel, or via gastric intubation.
13. A dietary composition comprising the single domain antibody according to any one of items 1-3, the fusion protein according to any one of items 4-5, the nucleic acid according to item 6, the vector according to item 7, and/or the host cell according to item 8, optionally wherein the dietary composition comprises one or more of prebiotics, probiotics, synbiotics, proteins, lipids, carbohydrates, vitamins, fibers, and/or nutrients, such as dietary minerals.
14. Use of the single domain antibody according to any one of items 1-3, the fusion protein according to any one of items 4-5, the nucleic acid according to item 6, the vector according to item 7, the host cell according to item 8, and/or the dietary composition according to item 13 as a food ingredient, as a food or beverage additive, or as food or beverage preservative.
15. A method for detecting Clostridium difficile, wherein the method comprises the steps of:
   a) providing a sample;
   b) contacting the sample with one of more single domain antibodies according to any one of items 1-3; and
   c) detecting the complex between the sample and the one or more single domain antibodies.

### Items 2

1. A single domain antibody which binds to Clostridium difficile toxin B.
2. The single domain antibody according to item 1, wherein the single domain antibody has one or more of the following features
   a) prevents and/or reduces Clostridium difficile toxin B cytotoxicity;
   b) prevents and/or reduces toxin activity of Clostridium difficile toxin B;
   c) prevents and/or reduces one or more Clostridium difficile-mediated symptoms, such as diarrhea, fever, hematochezia, and/or weight loss;
   d) reduces risk of Clostridium difficile infection;
   e) is stable in the gastrointestinal tract of a subject;
   f) is protease stable;
   g) is pH stable;
   h) is storage stable;
   i) is temperature stable; and/or
   j) improves the gut microbiome.
3. The single domain antibody according to any one of items 1-2, wherein the single domain antibody is selected from the group consisting of:
   a) a single domain antibody comprising:
      i. a complementary-determining region 1 (CDR1) comprising or consisting of SEQ ID NO: 1 or a variant thereof, wherein one or more amino acids have been altered, with the proviso that no more than 3 amino acids have been so altered, for example wherein 2, or 1 amino acids have been so altered; and
      ii. a complementary-determining region 2 (CDR2) comprising or consisting of SEQ ID NO: 2 or a variant thereof, wherein one or more amino acids have been altered, with the proviso that no more than 3 amino acids have been so altered, for example wherein 2, or 1 amino acids have been so altered; and
      iii. a complementary-determining region 3 (CDR3) comprising or consisting of SEQ ID NO: 3 or a variant thereof, wherein one or more amino acids have been altered, with the proviso that no more than 3 amino acids have been so altered, for example wherein 2, or 1 amino acids have been so altered;
   b) a single domain antibody comprising or consisting of the sequence as set forth in SEQ ID NO: 22, or a sequence having at least 90% sequence identity thereto; and
   c) a humanised version of the single domain antibody of a) or b).
4. The single domain antibody according to any one of items 1-2, wherein the single domain antibody is selected from the group consisting of:
   a) a single domain antibody comprising:
      i. a complementary-determining region 1 (CDR1) comprising or consisting of SEQ ID NO: 4 or a variant thereof, wherein one or more amino acids have been altered, with the proviso that no more than 3 amino acids have been so altered, for example wherein 2, or 1 amino acids have been so altered; and
      ii. a complementary-determining region 2 (CDR2) comprising or consisting of SEQ ID NO: 5 or a variant thereof, wherein one or more amino acids have been altered for another amino acid, with the proviso that no more than 3 amino acids have been so altered, for example wherein 2, or 1 amino acids have been so altered; and
      iii. a complementary-determining region 3 (CDR3) comprising or consisting of SEQ ID NO: 6 or a variant thereof, wherein one or more amino acids have been altered, with the proviso that no more than 3 amino acids have been so altered, for example wherein 2, or 1 amino acids have been so altered;
   b) a single domain antibody comprising or consisting of the sequence as set forth in SEQ ID NO: 23, or a sequence having at least 90% sequence identity thereto, and
   c) a humanised version of the single domain antibody of a) or b).
5. The single domain antibody according to any one of items 1-2, wherein the single domain antibody is selected from the group consisting of:
   a) a single domain antibody comprising:
      i. a complementary-determining region 1 (CDR1) comprising or consisting of SEQ ID NO: 7 or a variant thereof, wherein one or more amino acids have been altered, with the proviso that no more than 3 amino acids have been so altered, for example wherein 2, or 1 amino acids have been so altered; and
      ii. a complementary-determining region 2 (CDR2) comprising or consisting of SEQ ID NO: 8 or a variant thereof, wherein one or more amino acids have been altered, with the proviso that no more than 3 amino acids have been so altered, for example wherein 2, or 1 amino acids have been so altered; and
      iii. a complementary-determining region 3 (CDR3) comprising or consisting of SEQ ID NO: 9 or a variant thereof, wherein one or more amino acids have been altered, with the proviso that no more than 3 amino acids have been so altered, for example wherein 2, or 1 amino acids have been so altered;
   b) a single domain antibody comprising or consisting of the sequence as set forth in SEQ ID NO: 24, or a sequence having at least 90% sequence identity thereto, and
   c) a humanised version of the single domain antibody of a) or b).
6. The single domain antibody according to any one of items 1-2, wherein the single domain antibody is selected from the group consisting of:
   a) a single domain antibody comprising:
      i. a complementary-determining region 1 (CDR1) comprising or consisting of SEQ ID NO: 10 or a variant thereof, wherein one or more amino acids have been altered, with the proviso that no more than 3 amino acids have been so altered, for example wherein 2, or 1 amino acids have been so altered; and
      ii. a complementary-determining region 2 (CDR2) comprising or consisting of SEQ ID NO: 11 or a variant thereof, wherein one or more amino acids have been altered, with the proviso that no more than 3 amino acids have been so altered, for example wherein 2, or 1 amino acids have been so altered; and
      iii. a complementary-determining region 3 (CDR3) comprising or consisting of SEQ ID NO: 12 or a variant thereof, wherein one or more amino acids have been altered, with the proviso that no more than 3 amino acids have been so altered, for example wherein 2, or 1 amino acids have been so altered;
   b) a single domain antibody comprising or consisting of the sequence as set forth in SEQ ID NO: 25, or a sequence having at least 90% sequence identity thereto, and
   c) a humanised version of the single domain antibody of a) or b).
7. The single domain antibody according to any one of items 1-2, wherein the single domain antibody is selected from the group consisting of:
   a) a single domain antibody comprising:
      i. a complementary-determining region 1 (CDR1) comprising or consisting of SEQ ID NO: 13 or a variant thereof, wherein one or more amino acids have been altered, with the proviso that no more than 3 amino acids have been so altered, for example wherein 2, or 1 amino acids have been so altered; and
      ii. a complementary-determining region 2 (CDR2) comprising or consisting of SEQ ID NO: 14 or a variant thereof, wherein one or more amino acids have been altered, with the proviso that no more than 3 amino acids have been so altered, for example wherein 2, or 1 amino acids have been so altered; and
      iii. a complementary-determining region 3 (CDR3) comprising or consisting of SEQ ID NO: 15 or a variant thereof, wherein one or more amino acids have been altered, with the proviso that no more than 3 amino acids have been so altered, for example wherein 2, or 1 amino acids have been so altered;
   b) a single domain antibody comprising or consisting of the sequence as set forth in SEQ ID NO: 26, or a sequence having at least 90% sequence identity thereto, and
   c) a humanised version of the single domain antibody of a) or b).
8. The single domain antibody according to any one of items 1-2, wherein the single domain antibody is selected from the group consisting of:
   a) a single domain antibody comprising:
      i. a complementary-determining region 1 (CDR1) comprising or consisting of SEQ ID NO: 16 or a variant thereof, wherein one or more amino acids have been altered, with the proviso that no more than 3 amino acids have been so altered, for example wherein 2, or 1 amino acids have been so altered; and
      ii. a complementary-determining region 2 (CDR2) comprising or consisting of SEQ ID NO: 17 or a variant thereof, wherein one or more amino acids have been altered, with the proviso that no more than 3 amino acids have been so altered, for example wherein 2, or 1 amino acids have been so altered; and
      iii. a complementary-determining region 3 (CDR3) comprising or consisting of SEQ ID NO: 18 or a variant thereof, wherein one or more amino acids have been altered, with the proviso that no more than 3 amino acids have been so altered, for example wherein 2, or 1 amino acids have been so altered;
   b) a single domain antibody comprising or consisting of the sequence as set forth in SEQ ID NO: 27, or a sequence having at least 90% sequence identity thereto, and
   c) a humanised version of the single domain antibody of a) or b).
9. The single domain antibody according to any one of items 1-2, wherein the single domain antibody is selected from the group consisting of:
   a) a single domain antibody comprising:
      i. a complementary-determining region 1 (CDR1) comprising or consisting of SEQ ID NO: 19 or a variant thereof, wherein one or more amino acids have been altered, with the proviso that no more than 3 amino acids have been so altered, for example wherein 2, or 1 amino acids have been so altered; and
      ii. a complementary-determining region 2 (CDR2) comprising or consisting of SEQ ID NO: 20 or a variant thereof, wherein one or more amino acids have been altered, with the proviso that no more than 3 amino acids have been so altered, for example wherein 2, or 1 amino acids have been so altered; and
      iii. a complementary-determining region 3 (CDR3) comprising or consisting of SEQ ID NO: 21 or a variant thereof, wherein one or more amino acids have been altered, with the proviso that no more than 3 amino acids have been so altered, for example wherein 2, or 1 amino acids have been so altered;
   b) a single domain antibody comprising or consisting of the sequence as set forth in SEQ ID NO: 28, or a sequence having at least 90% sequence identity thereto, and
   c) a humanised version of the single domain antibody of a) or b).
10. The single domain antibody according to any one of the preceding items, wherein any sequence variance is outside the complementary-determining regions (CDRs).
11. The single domain antibody according to any one of the preceding items, wherein the alteration of one or more amino acids comprises a substitution, a deletion or an insertion.
12. The single domain antibody according item 11, wherein the alteration enhances the degree of aromatic and/or positively charged amino acids in the CDR(s).
13. The single domain antibody according to any one of items 11-12, wherein the alteration comprises a substitution and/or an insertion of one or more aromatic amino acids.
14. The single domain antibody according to item 13, wherein the aromatic amino acid is phenylalanine, tryptophan, tyrosine or histidine.
15. The single domain antibody according to any one of items 11-12, wherein the alteration comprises a substitution and/or an insertion of one or more positively charged amino acids.
16. The single domain antibody according to item 15, wherein the positively charged amino acid is lysine, arginine or histidine.
17. The single domain antibody according to any one of items 11-16, wherein the alteration is in CDR3.
18. The single domain antibody according to any one of the preceding items, wherein the single domain antibody is able to block the enzymatic activity of the glycosyltransferase domain of Clostridium difficile toxin B.
19. The single domain antibody according to item 18, wherein the glycosyltransferase domain has an amino acid sequence according to SEQ ID NO: 35.
20. The single domain antibody according to any one of the preceding items, wherein the single domain antibody comprises a detection label, such as a colorimetric, a fluorescent, a luminescent, a magnetic, or a paramagnetic label, or is biotinylated.
21. A fusion protein comprising a single domain antibody as defined in any one of the preceding items and one or more further single domain antibodies, and optionally one or more linkers.
22. The fusion protein according to item 21, wherein the fusion protein is a homodimer or a heterodimer.
23. The fusion protein according to any one of the items 21-22, wherein the one or more further single domain antibodies bind to Clostridium difficile toxin B and/or Clostridium difficile toxin A.
24. The fusion protein according to any one of items 21-23 comprising
   a) a single domain antibody as defined in item 3 and a further single domain antibody as defined in item 3;
   b) a single domain antibody as defined in item 3 and a further single domain antibody as defined in item 4;
   c) a single domain antibody as defined in item 3 and a further single domain antibody as defined in item 5;
   d) a single domain antibody as defined in item 3 and a further single domain antibody as defined in item 6;
   e) a single domain antibody as defined in item 3 and a further single domain antibody as defined in item 7;
   f) a single domain antibody as defined in item 3 and a further single domain antibody as defined in item 8;
   g) a single domain antibody as defined in item 3 and a further single domain antibody as defined in item 9;
   h) a single domain antibody as defined in item 4 and a further single domain antibody as defined in item 4;
   i) a single domain antibody as defined in item 4 and a further single domain antibody as defined in item 5;
   j) a single domain antibody as defined in item 4 and a further single domain antibody as defined in item 6;
   k) a single domain antibody as defined in item 4 and a further single domain antibody as defined in item 7;
   I) a single domain antibody as defined in item 4 and a further single domain antibody as defined in item 8;
   m) a single domain antibody as defined in item 4 and a further single domain antibody as defined in item 9;
   n) a single domain antibody as defined in item 5 and a further single domain antibody as defined in item 5;
   o) a single domain antibody as defined in item 5 and a further single domain antibody as defined in item 6;
   p) a single domain antibody as defined in item 5 and a further single domain antibody as defined in item 7;
   q) a single domain antibody as defined in item 5 and a further single domain antibody as defined in item 8;
   r) a single domain antibody as defined in item 5 and a further single domain antibody as defined in item 9;
   s) a single domain antibody as defined in item 6 and a further single domain antibody as defined in item 6;
   t) a single domain antibody as defined in item 6 and a further single domain antibody as defined in item 7;
   u) a single domain antibody as defined in item 6 and a further single domain antibody as defined in item 8;
   v) a single domain antibody as defined in item 6 and a further single domain antibody as defined in item 9;
   w) a single domain antibody as defined in item 7 and a further single domain antibody as defined in item 7;
   x) a single domain antibody as defined in item 7 and a further single domain antibody as defined in item 8;
   y) a single domain antibody as defined in item 7 and a further single domain antibody as defined in item 9;
   z) a single domain antibody as defined in item 8 and a further single domain antibody as defined in item 8;
   aa) a single domain antibody as defined in item 8 and a further single domain antibody as defined in item 9;
   bb) a single domain antibody as defined in item 9 and a further single domain antibody as defined in item 9.
25. The fusion protein according to any one of items 21-24, wherein the linker is a GS linker of the structure (GₓS)ₙ, where x may be a number between 1 to 10, preferably 2 to 5, and n refers to a number of repeats of the GₓS sequence, where n may be between 1 to 10, preferably 2 to 5.
26. The fusion protein according to item 25, wherein said GS linker is a GGGGS linker (SEQ ID NO: 29), a GGGGSGGGGS linker (SEQ ID NO: 30), a GGGGSGGGGSGGGGS linker (SEQ ID NO: 31), a GGGGSGGGGSGGGGSGGGGS linker (SEQ ID NO: 32), a GGGGSGGGGSGGGGSGGGGSGGGGS linker (SEQ ID NO: 33), or a GGGGSGGGGSGGGGSGGGGSGGGGSGGGGS linker (SEQ ID NO: 34).
27. An isolated nucleic acid molecule encoding the single domain antibody according to any one of items 1-20 or the fusion protein according to any one of items 21-26.
28. The isolated nucleic acid molecule according to item 27, wherein the nucleic acid molecule comprises or consists of SEQ ID NO: 36, SEQ ID NO: 37, SEQ ID NO: 38, SEQ ID NO: 39, SEQ ID NO: 40, SEQ ID NO: 41 and/or SEQ ID NO: 42.
29. The isolated nucleic acid molecule according to any one of items 27-28, wherein the nucleic acid molecule is codon-optimized for a host cell wherein said nucleic acid molecule is expressed.
30. An expression vector comprising the nucleic acid molecule according to any one of items 27-29.
31. The expression vector according to item 30, wherein the nucleic acid molecule is operably linked to one or more control sequences, such as an inducible promoter to direct its expression.
32. A recombinant host cell comprising the nucleic acid molecule according to any one of items 27-29 or the expression vector according to any one of items 30-31.
33. The recombinant host cell according to item 32, wherein the host cell is a bacterium, a plant, a fungus, such as a yeast, or a mammalian cell.
34. The recombinant host cell according to item 33, wherein the bacterium is a bacillus, such as a Bacillus licheniformis, Bacillus subtilis, Bacillus lactobacillus, Lactobacillus spp. or Bifidobacterium spp.
35. The recombinant host cell according to item 33, wherein the host cell is a yeast, such as a yeast selected from the genus of pichia, hansenula and saccharomyces.
36. The recombinant host cell according to item 33, wherein the fungus is selected from Aspergillus oryzae and Aspergillus niger.
37. A method of producing the single domain antibody according to any one of items 1-20 or the fusion protein according to any one of items 21-26, the method comprising culturing the host cell according to any one of items 32-36 under conditions wherein the single domain antibody or fusion protein is expressed.
38. The method according to item 37, wherein the method further comprises a step of purifying and/or isolating the single domain antibody molecule or fusion protein.
39. A composition comprising the single domain antibody according to any one of items 1-20, the fusion protein according to any one of items 21-26, the nucleic acid according to any one of items 27-29, the vector according to any one of items 30-31, and/or the host cell according to any one of items 32-36, optionally further comprising one or more excipients.
40. The composition according to item 39, wherein the composition comprises one or more further compounds selected from antibiotics, fecal matter transfer and/or monoclonal antibodies.
41. A single domain antibody according to any one of items 1-20, the fusion protein according to any one of items 21-26, the nucleic acid according to any one of items 27-29, the vector according to any one of items 30-31, the host cell according to any one of items 32-36 or the composition according to any one of items 39-40 for use as a medicament.
42. A single domain antibody according to any one of items 1-20, the fusion protein according to any one of items 21-26, the nucleic acid according to any one of items 27-29, the vector according to any one of items 30-31, the host cell according to any one of items 32-36 or the composition according to any one of items 39-40 for use in the prevention or treatment of Clostridium difficile infection in a subject.
43. The single domain antibody, the fusion protein, the nucleic acid, the vector, the host cell or the composition for use according to any one of items 41-42, wherein the subject is a human.
44. The single domain antibody, the fusion protein, the nucleic acid, the vector, the host cell or the composition for use according to any one of items 41-43, wherein the subject is an elderly subject, such as a subject of more than 65 years of age.
45. The single domain antibody, the fusion protein, the nucleic acid, the vector, the host cell or the composition for use according to any one of items 41-44, wherein said subject is an immunocompromised subject.
46. The single domain antibody, the fusion protein, the nucleic acid, the vector, the host cell or the composition for use according to any one of items 41-45, wherein the single domain antibody, the fusion protein, the nucleic acid, the vector, the host cell or the composition is administered enterally, such as orally, such as a food supplement, as a tablet or a gel, or via gastric intubation.
47. A method for prevention and/or treatment of Clostridium difficile infection in a subject in need thereof, said method comprising administering to the subject a single domain antibody according to any one of items 1-20, the fusion protein according to any one of items 21-26, the nucleic acid according to any one of items 27-29, the vector according to any one of items 30-31, the host cell according to any one of items 32-36 or the composition according to any one of items 39-40.
48. A method for reducing the risk of Clostridium difficile infection in a subject in need thereof, said method comprising administering to the subject a single domain antibody according to any one of items 1-20, the fusion protein according to any one of items 21-26, the nucleic acid according to any one of items 27-29, the vector according to any one of items 30-31, the host cell according to any one of items 32-36 or the composition according to any one of items 39-40.
49. A dietary composition comprising the single domain antibody according to any one of items 1-20, the fusion protein according to any one of items 21-26, the nucleic acid according to any one of items 27-29, the vector according to any one of items 30-31, and/or the host cell according to any one of items 32-36.
50. The dietary composition according to item 49, wherein said dietary composition comprises one or more of prebiotics, probiotics, synbiotics, proteins, lipids, carbohydrates, vitamins, fibers, and/or nutrients, such as dietary minerals.
51. Use of the single domain antibody according to any one of items 1-20, the fusion protein according to any one of items 21-26, the nucleic acid according to any one of items 27-29, the vector according to any one of items 30-31, the host cell according to any one of items 32-36 and/or the dietary composition according to any one of items 49-50 as a food ingredient, as a food or beverage additive, or as food or beverage preservative.
52. A method for detecting Clostridium difficile, wherein the method comprises the steps of:
   a) providing a sample;
   b) contacting the sample with one of more single domain antibodies according to any one of the items 1-20; and
   c) detecting the complex between the sample and the one or more single domain antibodies.
53. The method according to item 52 wherein step b) further comprises a step of washing the sample, thereby removing any unbound antibody.
54. The method according to any one of items 52-53 wherein the method comprises detecting the complex of step c) by western blotting, ELISA, LFA, microscopy, flow cytometry; TRF, or immunocytochemistry.

### EXAMPLES

### Example 1. Screening of Single-Domain Antibodies

The in vitro binding capacity of various single-domain antibody monomers to recombinant TcdB-GT toxin was tested by a time-resolved fluorescence normalized assay (DELFIA). Briefly, black 96-well immunoblot plates were coated with 2.5 μl/ml of anti-FLAG M2 antibody (SIGMA #F3165) in phosphate buffered saline (PBS) pH 7.4 overnight (O/N) at 4° C. After blocking with milk (3% in PBS) for 1 hour at room temperature, the supernatant of single-domain antibody cultures was added at OD600=10 in 6% milk-PBS and incubated at room temperature for 1 hour. The supernatant of single-domain antibody controls 5D and E3 reported by Yang Z. et al. 2014. JID. 210 (6). 964-972 (DOI: 10.1093/infdis/jiu196), were used for comparison.

According to Yang et al. 2014, the sdAbs 5D and E3 are potent TcdB-neutralizing VHH's targeting the glucosyltransferase domain.

After washing, biotinylated toxin (recombinant TcdB-GT) was added at 25 nM in 3% milk-PBS and incubated for 1 h at room temperature. After washing, streptavidin-conjugated europium (Perkin Elmer, #1244-360) was added dilute 1/500 in DELFIA assay buffer (Perkin Elmer #1244-111) and incubated for 30 minutes at room temperature. After washing, europium fluorescence was activated using DELFIA enhancement solution (Perkin Elmer #4001-0010). Fluorescence intensity was determined using a microplate reader measuring emission at 615 nm.

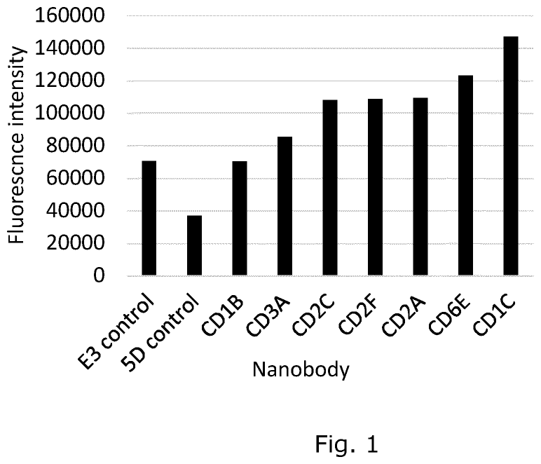

A total of seven single-domain antibodies were selected based on the binding properties against the recombinant TcdB-GT toxin (FIG. 1).

The inventors of the present disclosure demonstrate how at least some of the single-domain antibodies screened, showed a higher fluorescence intensity compared with the single-domain antibody controls E3 and 5D (FIG. 1). The higher intensity demonstrates an enhanced binding efficacy of the single-domain antibodies against the recombinant toxin TcdB-GT.

Moreover, the values of the fluorescence intensity show that at least some of the single-domain antibodies selected have better binding to the recombinant TcdB-GT toxin compared with the controls E3 and 5D.

### Example 2. Single-Domain Antibody Blocking Activity of the Glycosyltransferase Domain

An assay of blocking of the enzymatic activity of the Glycosyltransferase domain (GT) of the TcdB toxin using single-domain antibodies was designed. Briefly: two concentrations of each single-domain antibody selected was incubated with the recombinant TcdB-GT toxin domain in phosphate buffered saline (PBS) pH 7.4 solution for 1 hour. After incubation, the samples were transferred to a 96-well plate, clear, flat bottom, non-binding surface (Cayman chemical company #400014) and mixed with assay buffer (50 mM Hepes pH 7.4, 150 mM KCl, 5 mM MgCl2. 0.5 nM NADH). The reaction components were then added: 0.5 mM phosphoenolpyruvate (PEP), 1 and 1.5 units of pyruvate kinase/lactate dehydrogenase (PK/LDH) and 2 nM UDP-glucose. NADH consumption due to the enzymatic reaction of glucosyltransferase was monitored by measuring time points every 30 seconds on a plate reader at 340 nm.

The data were analyzed calculating the slope of the kinetic curve and translated into percentage of enzymatic activity without and with single-domain antibody.

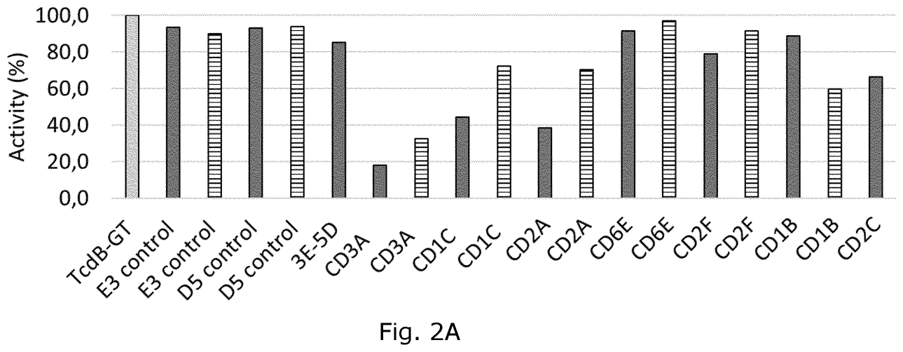

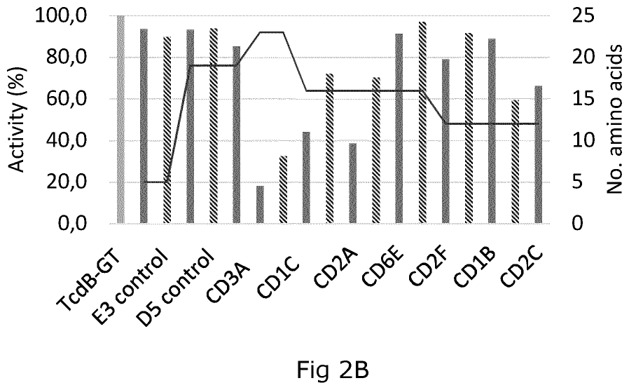

All single-domain antibodies demonstrated a better blocking capacity compared with the control single-domain antibodies used, since they decreased recombinant TcdB-GT activity below the values of the controls (FIG. 2 and FIG. 2b).

### Example 3. CDR Homology Analysis (or Aminoacidic Sequence Analysis)

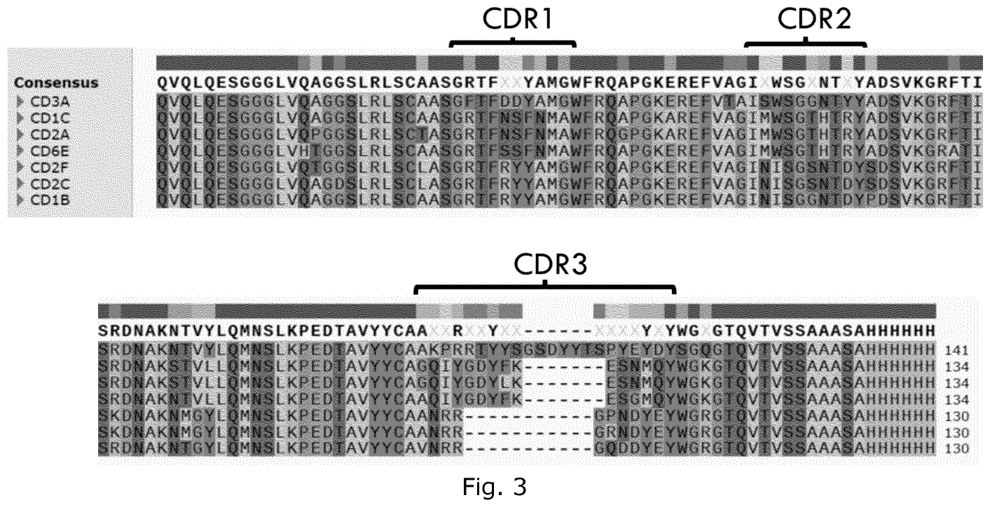

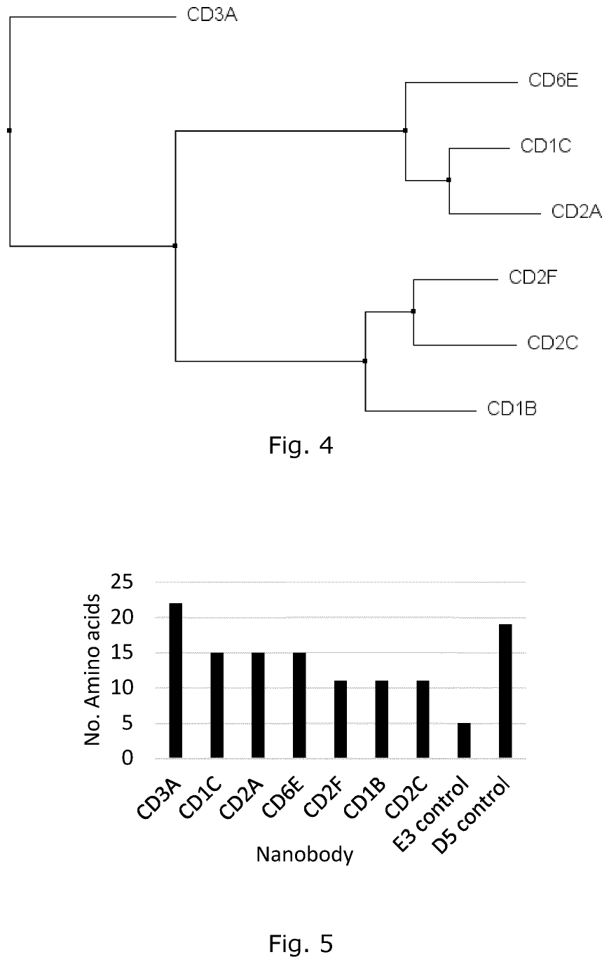

The amino acid sequence of the selected single-domain antibody was analyzed to create a homology-based classification. The analysis was performed using Clustal Omega multiple sequence alignment (guide trees and hidden Markov model (HMM) profile-profile) (FIG. 3 and FIG. 4).

The main differences in the single-domain antibody sequences were found in the CDR3s. In the selected single-domain antibody, the relationship between the length of CDR3s and the ability to block the activity of the recombinant toxin TcdB-GT was clear. Family one, containing only CD3A single-domain antibody with a CDR3 length of 23 residues showed the best neutralizing activity, followed by single-domain antibodies from the group 2 (CD6E, CD1C and CD2A) with CDR3 length of 16 residues. Finally, the third group (CD2F, CD2C and CD1C) had the shortest CDR3 of 12 residues (FIGS. 2b and 5).

The inventors found that the cluster of the single-domain antibodies in three families is correlated with their blocking activity. The first clade, single-domain antibody CD3A, has the best blocking activity followed by the single-domain antibodies from the second group (CD6E, CD1C and CD2A) and the third clade (CD2F, CD2C and CD1B).

Analyzing the biophysical properties of the amino acids in CDR3, it was found that the CD3A single-domain antibody has a higher content of aromatic and positively charged residues. These types of amino acids are in lower proportion in group 2 (single-domain antibodies: CD6E, CD1C and CD2A) and to an even lesser extent in group 3 (single-domain antibodies: CD2F, CD2C and CD1B). Thus, it may be beneficial to increase the content of aromatic and positively charged residues in the CDRs, particularly in CDR3.

### Example 4. Affinity Measurement

Measurement of the equilibrium dissociation constant (K_{D}), denominated as affinity, of the single domain antibodies were performed by bio-layer interferometry (BLI) on OctetRED96 system (Sartorius). Briefly, 50 mM of biotinylated recombinant TcdB-GT toxin was used for capture to a streptavidin (SA) biosensor (Fortebio Cat. 10-0009). The ligand-loaded biosensors were immersed in dilutions of each of the single-domain antibodies in 1× kinetic buffer (Sartorius Cat. 181105) ranging from 2.5 to 150 nM. The curves obtained were analyzed by the Octet Analysis studio Satorius software for estimation of kinetic parameters; ka, kd, local, and global KD (Table 1, below).

The present example demonstrates that two single domain antibodies had high binding affinity, in the picomolar (pM) range, to TcdB-GT toxin. Compared to the control single domain antibody E3 (Yang et al., 2014), the K_{D} values obtained for the single domain antibodies selected were lower (Table 1). To exemplify, the K_{D} for CD1C is at least 30-fold higher than the reported K_{D} for the control.

TABLE 1
Kinetic parameters of the two single
domain antibodies obtained by BLI.

| sdAb | kₐ (1/Ms) | k_{d} (1/s) | K_{D} (pM) |
|---|---|---|---|
| CD3A | 2.2e⁵ | 1e⁻⁷ | 21 |
| CD1C | 7e⁶ | 1e⁻⁷ | <1 |
| E3 (control)* | 2.95e⁶ | 9.4e⁻⁵ | 30 |

*Relative affinity reported in Yang et al., 2014
kₐ: association rate constant value.
k_{d}: dissociation rate constant vale.
K_{D}: equilibrium dissociation constant

### Example 5. Single-Domain Antibody Blocking Activity of the Native Glycosyltransferase Domain

An assay of blocking of the enzymatic activity of the glycosyltransferase domain (GT) from the native TcdB toxin using single-domain antibodies was designed. Briefly: the single domains were mixed with the native TcdB toxin (NAC cat. CDB-TNL-100) in 1×PBS buffer in a ratio of 20:1 and incubated at 37° C. for 30 min. After incubation the TedB toxin was submitted to cleavage in vitro of the GT domain by adding 100 UM of inositol hexaphosphoric acid (InsP6) (Sigma-aldrich: Cat. 68388) per 1 μg of toxin. The mixture was incubated for 1 hour at 37° C. After incubation, the sample was transferred to an Amicon ultra centrifugal filter 3K (Milipore Cat. UFC501096) and 2 volumes of 1×PBS were added. The sample was centrifugated for 2 min at 3000 rcf to remove the InsP6. The samples were transferred to a 96-well plate with a clear, flat bottom, and non-binding surface (Cayman chemical company #400014) and mixed with assay buffer (50 mM 10 Hepes pH 7.4, 150 mM KCl, 5 mM MgCl2. 0.5 nM NADH). The reaction components were then added: 0.5 mM phosphoenolpyruvate (PEP), 1 and 1.5 units of pyruvate kinase/lactate dehydrogenase (PK/LDH) and 2 nM UDP-glucose. In this assay, glucosyltransferase catalyzes UDP-glucose, and the product of this reaction is then taken up for the next reaction. The chain of coupled reactions leads to the oxidation of NADH to NAD. The NADH consumption can be measured at 340 nm absorption. The reaction was monitored every 30 seconds for 2 hours on a plate reader.

The data were analyzed by calculating the slope of the kinetic curve and translated into percentage enzyme activity, where the value obtained with TcdB-GT alone was taken as 100% activity and compared with the values obtained in the presence of the single domain antibody.

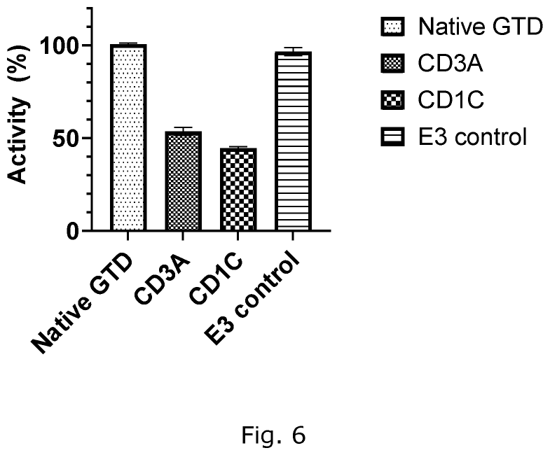

In this assay, the selected single domain antibodies, CD3A and CD1C, were able to reduce the activity of the native TcdB-GT by approximately 50-60% (see FIG. 6).

As can be seen in FIG. 6, two single domain antibodies according to the invention, CD3A and CD1C, reduce the activity of the TedB-GT toxin activity more efficiently than control E3 (Yang et al., 2014).

### Example 6. pH Functional Stability

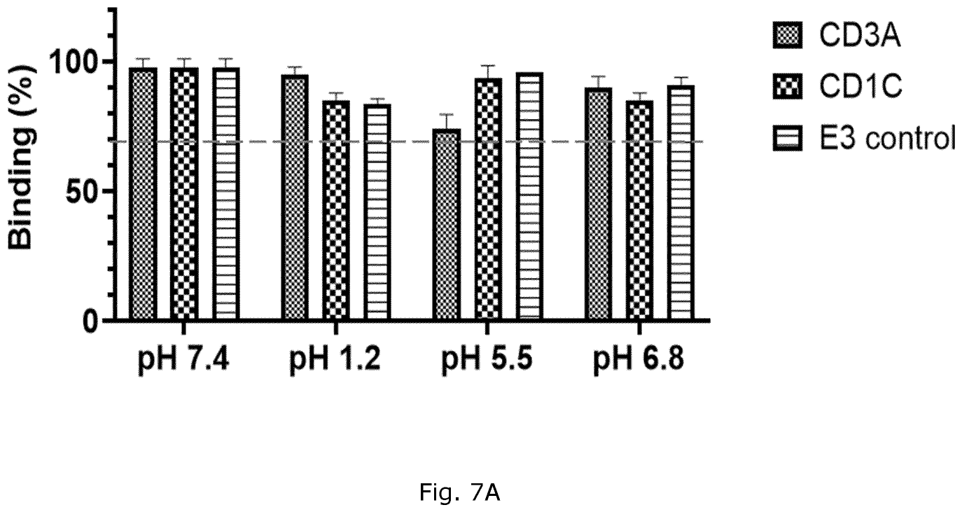

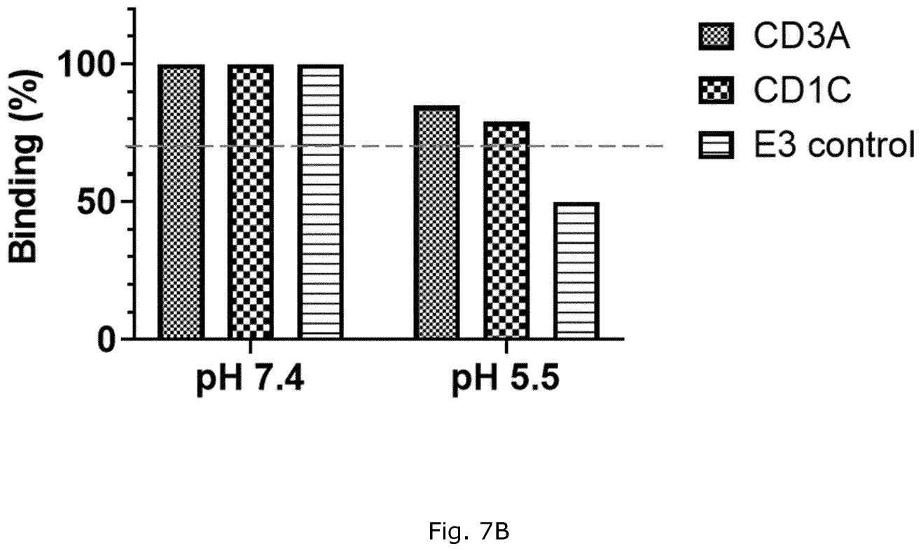

The binding capacity of the single domain antibodies to the recombinant and native TcdB-GT toxin in different physiologically relevant pHs was tested by Enzyme-linked immunosorbent assay (ELISA). Briefly, 96-well high binding plates (MaxiSorp Nunc, Cat. 44-2404-21) were coated with 15 μg/ml of recombinant TcdB-GT or 10 μg/ml of native TcdB toxin overnight at 4° C. The plates were washed 3 times with 1×PBS+0.1% Tween followed by 3 times 1× PBS and incubated with blocking solution of 3% nonfat dry milk in 1×PBS for 1 hour at 37° C. In parallel, dilutions of 20 μg/ml of each single domain antibody were prepare in solutions with different pHs; 1× PBS pH 7.4, Simulated gastric solution (SGF) NaCl 35 mM pH 1.2, Simulated intestinal fluid (SIF) 50 mM KH₂PO₄ PH 6.8 and MES 50 mM pH 5.5 and incubated for at least 60 min at 37° C. The plates were washed 3 times with 1×PBS+0.1% Tween followed by 3 times with 1×PBS. Then, treated single domain antibodies were added to the wells and incubated for 1 hour at 37° C. After incubation, the plates were washed 3 times with 1×PBS+0.1% Tween followed by 3 times with 1×PBS. Mouse anti-flag tag horseradish peroxidase (HRP) conjugated antibody diluted 1:20,000 in blocking solutions was added to the wells and incubated for 1 hour at 37° C. After incubation, the plates were washed and the peroxidase substrate 3,3′,5,5,5-tetramethylbenzidine (TMB) in peroxide solution was added and incubated for 10-40 minutes until the colorimetric reaction developed. The reaction was stopped with 1 M H₂SO₄ and read at 450 nm on a plate reader. The values were analyzed and represented as percentage of binding by calculating and using the mean of the controls (PBS 7.4) as a reference of 100% binding to compare the tested samples (FIGS. 7A and 7B).

This experiment demonstrated how two examples of single domain antibodies according to the present disclosure are stable and maintain above 70% binding to the recombinant TcdB-GT and native TcdB toxin after being subjected in buffers that mimic gastric conditions—with emphasis on the binding at pH 5.5, which is a relevant condition during the internalization of the toxin to the cell (FIG. 7A-B) (Chandrasekran and Lacy. 2017).

### Example 7. Thermostability

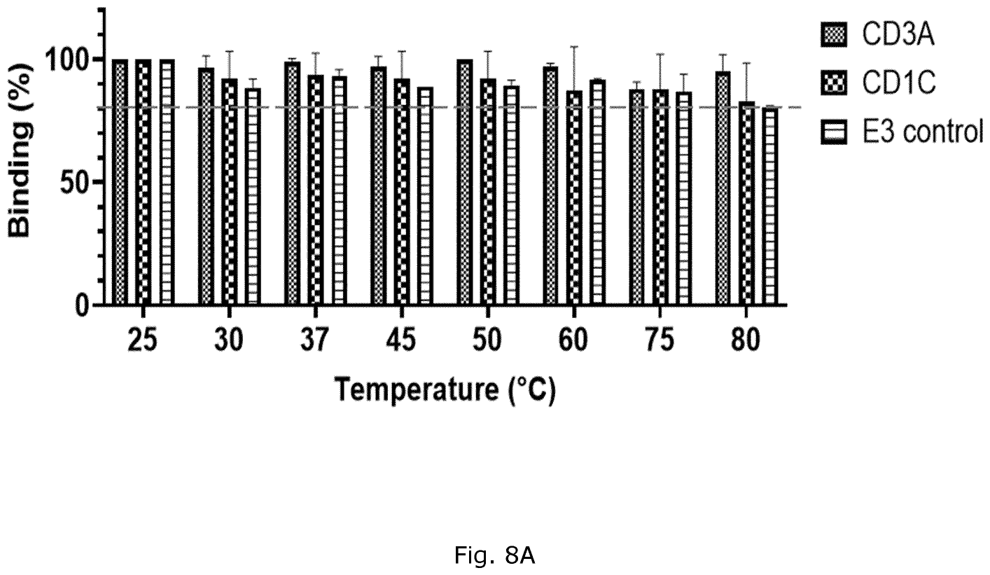

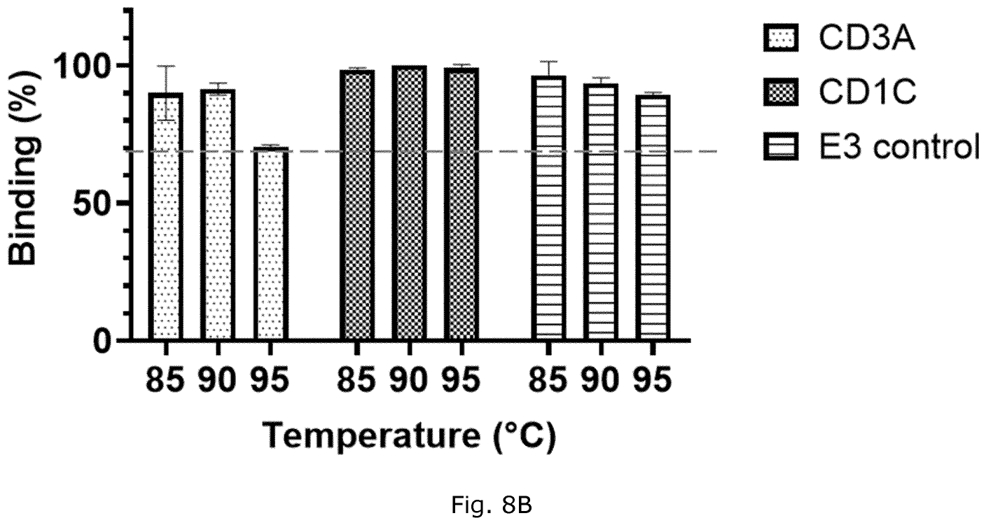

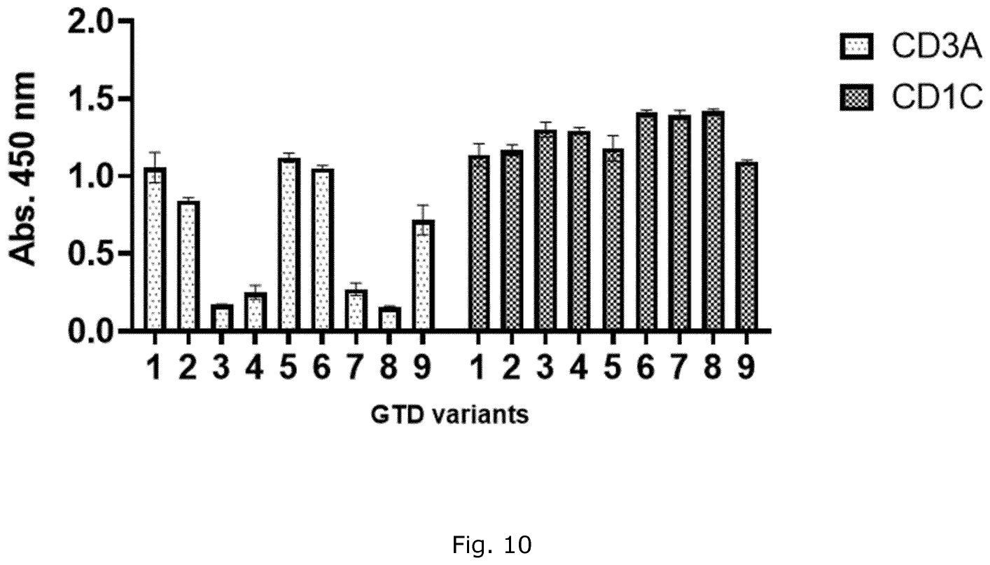

The stability and functionality (binding) of single domain antibodies after being subjected to heat stress was tested. Briefly, each single domain antibody at a concentration of 20 μg/ml was incubated for 1 hour at 25, 30, 37, 45, 50, 60, 75 and 80° C. (FIG. 8A) and 10 sec at 85, 90 and 95° C. (FIG. 8B). After the incubation the single domains were submitted to a binding assay to the recombinant TcdB-GT by ELISA. 96-well high binding plates (MaxiSorp Nunc, Cat. 44-2404-21) were coated with 15 μg/ml of recombinant TcdB-GT toxin overnight at 4° C. The plates were washed 3 times with 1×PBS+0.1% Tween followed by 3 times with 1×PBS and incubated with blocking solution of 3% nonfat dry milk in 1×PBS for 1 hour at 37° C. The plates were washed 3 times with 1×PBS+0.1% Tween followed by 3 times with 1×PBS and incubated individually with the single domain antibodies temperature treated for 1 hour at 37° C. After incubation, the plates were washed 3 times with 1×PBS+0.1% Tween followed by 3 times with 1×PBS. Mouse anti-flag tag horseradish peroxidase (HRP) conjugated antibody diluted 1:20,000 in blocking solutions was added to the wells and incubated for 1 hour at 37° C. After incubation, the plates were washed as above and the peroxidase substrate 3,3′,5,5,5-tetramethylbenzidine (TMB) in peroxide solution was added and incubated for 10-40 minutes until the colorimetric reaction developed. The reaction was stopped with 1 M H₂SO₄ and read at 450 nm on a plate reader. The values were analyzed and represented as percentage of binding by calculating and using the mean of the controls (25° C.) as a reference of 100% binding to compare the tested samples.

The experiment demonstrated how two examples of single domain antibodies according to the present disclosure maintained about 80% of target binding even after incubation for 1 hour at 80° C. (FIG. 8A). Under harsher conditions, the CD3A, lost around 30% of binding at the highest temperature, however the CD1C maintained the same binding capacity (FIG. 8B).

These results demonstrate that the single domain antibodies were thermostable and maintained binding to the target after being submitted to a broad range of temperatures.

### Example 8. Shelf Stability in Liquid

Single domain antibodies were tested for shelf stability in liquid conditions at 4° C. Briefly, 25 μg of each single domain antibody was diluted in 1×PBS (pH 7.4) or 1% skimmed milk and submitted to a heating process by incubating the samples at 63° C. for 30 minutes and stored at 4° C. for 7 days. After 7 days, binding was tested by ELISA. 96 well high binding plates (MaxiSorp Nunc, Cat. 44-2404-21) were coated with 15 μg/ml of recombinant TcdB-GT toxin variants overnight at 4° C. The plates were washed 3 times with 1×PBS+0.1% Tween followed by 3 times with 1×PBS and incubated with blocking solution of 3% nonfat dry milk in 1×PBS for 1 hour at 37° C. The plates were washed 3 times with 1×PBS+0.1% Tween followed by 3 times with 1×PBS. Then, single domain antibody was added to the wells and incubated with the TcdB-GT toxin for 1 hour at 37° C. After incubation, the plates were washed 3 times with 1×PBS+0.1% Tween followed by 3 times with 1×PBS. Mouse anti-flag tag horseradish peroxidase (HRP) conjugated antibody diluted 1:20,000 in blocking solutions was added to the wells and incubated for 1 hour at 37° C. After incubation, the plates were washed as above and the peroxidase substrate 3,3′,5,5,5-tetramethylbenzidine (TMB) in peroxide solution was added and incubated for 10-40 minutes until the colorimetric reaction developed. The reaction was stopped with 1 M H₂SO₄ and read at 450 nm on a plate reader. The values were analyzed and represented as percentage of binding by calculating and using the mean of the controls (PBS) as a reference of 100% binding to compare the tested samples.

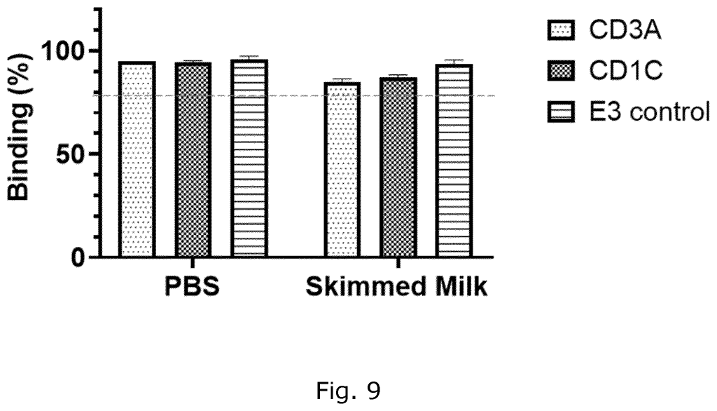

As can be seen in FIG. 9, this experiment demonstrated how two single domain antibodies according to the present disclosure are stable and maintain about 80% of binding to the target after being stored at 4° C. for at least 7 days in liquids, such as milk or 1× PBS.

### Example 9. Cross Reactivity with TcdB-GT Toxin Variants

The binding capacity of the single domain antibodies against different TcdB-GT toxin variants was tested. Briefly, nine different TcdB-GT toxins were recombinantly produced and purified. 96 well high binding plates (MaxiSorp Nunc, Cat. 44-2404-21) were coated with 15 μg/ml of recombinant TcdB-GT toxin variants overnight at 4° C. The plates were washed 3 times with 1×PBS+0.1% Tween followed by 3 times with 1×PBS and incubated with blocking solution of 3% nonfat dry milk in 1×PBS for 1 hour at 37° C. The plates were washed 3 times with 1×PBS+0.1% Tween followed by 3 times with 1× PBS. Each single domain antibody was incubated with the different TcdB-GT toxin variants for 1 hour at 37° C. After incubation, the plates were washed 3 times with 1×PBS+0.1% Tween followed by 3 times with 1×PBS. Mouse anti-flag tag horseradish peroxidase (HRP) conjugated antibody diluted 1:20,000 in blocking solutions was added to the wells and incubated for 1 hour at 37° C. After incubation, the plates were washed as above and the peroxidase substrate 3,3′,5,5,5-tetramethylbenzidine (TMB) in peroxide solution was added and incubated for 10-40 minutes until the colorimetric reaction developed. The reaction was stopped with 1 M H₂SO₄ and read at 450 nm on a plate reader (FIG. 10).

It was observed that one of the single domain antibodies (CD1C) bound to a broad range of variants compared to the other tested single domain antibody (CD3A), that bound to 5 out 9 TcdB-GT variants.

Due to the observed direct correlation of binding to recombinant TcdB-GT and thus to native TcdB-GT toxin (FIG. 7A-B), the results observed in this cross-reactivity experiment (FIG. 10) suggested that both single domain antibodies can recognize different TcdB variants.

### Sequence Overview

| SEQ ID | | |
|---|---|---|
| NO: | Name | Sequence |
| 1 | CD3A-CDR1 | GFTFDDYAMG |
| 2 | CD3A-CDR2 | ISWSGGNTYY |
| 3 | CD3A-CDR3 | AAKPRRTYYSGSDYYTSPYEYDY |
| 4 | CD1C-CDR1 | GRTFNSFNMA |
| 5 | CD1C-CDR2 | IMWSGTHTRY |
| 6 | CD1C-CDR3 | AGQIYGDYFKESNMQY |
| 7 | CD2A-CDR1 | GRTFNSFNMA |
| 8 | CD2A-CDR2 | IMWSGTHTRY |
| 9 | CD2A-CDR3 | AGQIYGDYLKESNMQY |
| 10 | CD6E-CDR1 | GRTFSSFNMA |
| 11 | CD6E-CDR2 | IMWSGTHTRY |
| 12 | CD6E-CDR3 | AAQIYGDYFKESGMQY |
| 13 | CD2F-CDR1 | GRTFRYYAMG |
| 14 | CD2F-CDR2 | INISGSNTDY |
| 15 | CD2F-CDR3 | AANRRGPNDYEY |
| 16 | CD2C-CDR1 | GRTFRYYAMG |
| 17 | CD2C-CDR2 | INISGSNTDY |
| 18 | CD2C-CDR3 | AANRRGRNDYEY |
| 19 | CD1B-CDR1 | GRTFRYYAMG |
| 20 | CD1B-CDR2 | INISGGNTDY |
| 21 | CD1B-CDR3 | AVNRRGQDDYEY |
| 22 | CD3A-Protein | QVQLQESGGGLVQAGGSLRLSCAASGFTFDDYAMG |
| | | WFRQAPGKEREFVTAISWSGGNTYYADSVKGRFTIS |
| | | RDNAKNTVYLQMNSLKPEDTAVYYCAAKPRRTYYSG |
| | | SDYYTSPYEYDYSGQGTQVTVSS |
| 23 | CD1C-Protein | QVQLQESGGGLVQAGGSLRLSCAASGRTFNSFNMA |
| | | WFRQAPGKAREFVAGIMWSGTHTRYADSVKGRFTIS |
| | | RDNAKSTVLLQMNSLKPEDTAVYYCAGQIYGDYFKES |
| | | NMQYWGKGTQVTVSS |
| 24 | CD2A-Protein | QVQLQESGGGLVQPGGSLRLSCTASGRTFNSFNMA |
| | | WFRQGPGKAREFVAGIMWSGTHTRYADSVKGRFTIS |
| | | RDNAKSTVLLQMNSLKPEDTAVYYCAGQIYGDYLKES |
| | | NMQYWGKGTQVTVSS |
| 25 | CD6E-Protein | QVQLQESGGGLVHTGGSLRLSCAASGRTFSSFNMA |
| | | WFRQAPGKEREFVAGIMWSGTHTRYADSVKGRATIS |
| | | RDNAKNTVLLQMNSLKPEDTAVYYCAAQIYGDYFKES |
| | | GMQYWGKGTQVTVSS |
| 26 | CD2F-Protein | QVQLQESGGGLVQTGGSLRLSCLASGRTFRYYAMG |
| | | WFRQAPGKEREFVAGINISGSNTDYSDSVKGRFTISK |
| | | DNAKNMGYLQMNSLKPEDTAVYYCAANRRGPNDYE |
| | | YWGRGTQVTVSS |
| 27 | CD2C-Protein | QVQLQESGGGLVQAGDSLRLSCLASGRTFRYYAMG |
| | | WFRQAPGKEREFVAGINISGSNTDYSDSVKGRFTISK |
| | | DNAKNMGYLQMNSLKPEDTAVYYCAANRRGRNDYE |
| | | YWGRGTQVTVSS |
| 28 | CD1B-Protein | QVQLQESGGGLVQAGGSLRLSCAASGRTFRYYAMG |
| | | WFRQAPGKEREFVAGINISGGNTDYPDSVKGRFTISR |
| | | DNAKNTGYLQMNSLKPEDTAVYYCAVNRRGQDDYE |
| | | YWGRGTQVTVSS |
| 29 | Linker- | GGGGS |
| | (GGGGS)₁ | |
| 30 | Linker- | GGGGSGGGGS |
| | (GGGGS)₂ | |
| 31 | Linker- | GGGGSGGGGSGGGGS |
| | (GGGGS)₃ | |
| 32 | Linker- | GGGGSGGGGSGGGGSGGGGS |
| | (GGGGS)₄ | |
| 33 | Linker- | GGGGSGGGGSGGGGSGGGGSGGGGS |
| | (GGGGS)₅ | |
| 34 | Linker- | GGGGSGGGGSGGGGSGGGGSGGGGSGGGGS |
| | (GGGGS)₆ | |
| 35 | GT domain of | ATGTCTTTAGTTAACCGCAAGCAATTAGAAAAGATG |
| | C. difficile | GCTAACGTGCGGTTCCGGACTCAAGAGGACGAGTA |
| | TcdB | CGTTGCGATCTTAGACGCACTGGAAGAGTATCATA |
| | | ATATGAGTGAGAATACTGTAGTTGAGAAGTATCTGA |
| | | AGTTAAAGGACATAAACTCGTTGACAGATATATACA |
| | | TTGACACTTACAAAAAATCTGGAAGAAACAAGGCAC |
| | | TGAAGAAGTTCAAAGAGTATTTGGTTACAGAGGTAC |
| | | TGGAGCTGAAGAACAATAACCTGACGCCAGTCGAA |
| | | AAGAATTTGCACTTTGTGTGGATTGGAGGACAGATT |
| | | AATGACACGGCGATAAACTACATAAATCAATGGAAG |
| | | GACGTCAACTCCGACTACAATGTCAATGTCTTTTAT |
| | | GACTCCAATGCGTTTTTAATTAATACCCTGAAGAAA |
| | | ACAGTTGTTGAAAGTGCTATAAATGATACACTTGAG |
| | | TCCTTCCGTGAGAACTTGAACGACCCCCGGTTCGA |
| | | TTATAATAAGTTCTTTCGCAAAAGAATGGAGATTATA |
| | | TACGACAAACAAAAGAACTTCATTAATTACTACAAG |
| | | GCACAACGGGAAGAAAACCCGGAGTTAATAATAGA |
| | | TGATATAGTCAAGACCTACTTGTCTAATGAATATTC |
| | | CAAGGAAATAGACGAGTTAAATACTTATATTGAGGA |
| | | GAGTCTGAATAAAATCACTCAAAACTCGGGAAATGA |
| | | TGTGAGAAATTTTGAGGAGTTTAAGAATGGGGAGA |
| | | GCTTTAATCTTTACGAGCAGGAGCTGGTAGAGCGT |
| | | TGGAATCTTGCTGCGGCGTCAGACATTTTGCGGAT |
| | | CAGCGCCCTTAAGGAGATAGGGGGAATGTATTTAG |
| | | ACGTAGACATGTTGCCAGGGATTCAGCCCGACCTG |
| | | TTTGAATCCATCGAAAAACCGTCAAGCGTCACGGT |
| | | CGACTTTTGGGAGATGACCAAACTTGAGGCTATAAT |
| | | GAAGTACAAGGAATATATACCAGAGTATACCAGTGA |
| | | GCATTTTGACATGCTTGATGAAGAAGTTCAGTCATC |
| | | ATTTGAGTCAGTACTTGCAAGTAAATCTGACAAGAG |
| | | TGAGATATTTAGCTCGTTAGGCGATATGGAGGCTT |
| | | CACCACTTGAGGTCAAGATCGCTTTCAATAGCAAA |
| | | GGAATCATCAACCAGGGATTAATTTCTGTTAAAGAC |
| | | TCCTACTGTTCTAACTTGATAGTCAAACAAATTGAA |
| | | AACCGTTACAAGATACTGAATAATAGTCTTAACCCA |
| | | GCCATTAGCGAGGACAACGACTTTAACACGACCAC |
| | | TAACACATTCATCGATTCAATTATGGCCGAAGCAAA |
| | | TGCGGACAACGGTCGGTTCATGATGGAATTAGGGA |
| | | AGTACCTGCGTGTGGGCTTCTTCCCGGATGTGAAG |
| | | ACAACCATAAACCTGAGTGGTCCGGAAGCCTACGC |
| | | AGCGGCATATCAAGACCTTTTGATGTTTAAAGAAGG |
| | | ATCTATGAACATTCACTTAATAGAGGCCGACCTGCG |
| | | TAACTTCGAAATCTCTAAAACGAATATATCGCAGTC |
| | | AACGGAACAGGAGATGGCATCCCTGTGGTCTTTCG |
| | | ACGACGCTCGCGCGAAAGCACAATTCGAGGAATAC |
| | | AAGCGCAATTATTTCGAAGGTAGTCTGGGGGAAGA |
| | | TGACAATTTAGACTTTAGCCAGAATATCGTGGTGGA |
| | | CAAAGAGTATTTGTTGGAAAAGATCTCAAGTTTAGC |
| | | CCGGAGCTCGGAGCGCGGATATATACACTACATAG |
| | | TGCAGTTACAAGGGGACAAAATTTCATACGAGGCG |
| | | GCATGTAATCTTTTTGCAAAGACGCCCTACGATAGC |
| | | GTGCTGTTCCAGAAAAACATCGAGGACTCGGAGAT |
| | | AGCGTATTACTACAATCCAGGTGATGGGGAAATAC |
| | | AGGAGATAGATAAGTACAAGATACCATCGATTATCT |
| | | CCGATCGTCCCAAGATCAAGTTGACCTTTATCGGT |
| | | CACGGCAAGGATGAATTCAACACCGACATTTTTGC |
| | | AGGTTTTGACGTTGATTCGCTGTCCACAGAAATTGA |
| | | GGCCGCGATCGACCTGGCGAAGGAGGATATATCC |
| | | CCTAAATCTATCGAGATTAACTTACTTGGGTGTAAC |
| | | ATGTTCTCCTACTCTATTAACGTCGAGGAAACGTAT |
| | | CCCGGTAAGCTGTTATTAAAAGTCAAAGACAAAATA |
| | | TCCGAACTGATGCCTAGTATCAGCCAGGACTCAAT |
| | | TATCGTCTCCGCGAACCAGTATGAAGTTCGTATAAA |
| | | CTCAGAGGGTCGTCGCGAATTATTAGATCACAGCG |
| | | GGGAATGGATTAATAAGGAGGAGAGCATAATAAAG |
| | | GACATCTCCTCGAAAGAGTATATATCATTCAATCCG |
| | | AAGGAAAATAAGATAACAGTTAAGAGTAAGAACTTA |
| | | CCAGAATTGTCAACCCTTCTTCAGGAGATTCGCAAC |
| 36 | CD3A-DNA | CAGGTGCAGCTGCAGGAGTCTGGGGGAGGCTTGG |
| | | TGCAGGCTGGGGGGTCTCTGAGACTCTCCTGTGCA |
| | | GCCTCTGGATTCACTTTCGATGATTATGCCATGGG |
| | | CTGGTTCCGCCAGGCTCCAGGGAAGGAGCGTGAG |
| | | TTTGTAACAGCTATTAGCTGGAGTGGTGGTAACACA |
| | | TACTATGCAGACTCCGTGAAGGGCCGATTCACCAT |
| | | CTCCAGAGACAACGCCAAGAACACGGTGTATCTGC |
| | | AAATGAACAGCCTGAAACCTGAGGACACGGCCGTT |
| | | TATTACTGTGCAGCCAAGCCCCGTCGCACATACTA |
| | | TAGTGGTAGTGACTACTACACGAGCCCATATGAGT |
| | | ATGACTACTCTGGCCAGGGGACCCAGGTCACCGTC |
| | | TCCTCA |
| 37 | CD1C-DNA | CAGGTGCAGCTGCAGGAGTCTGGGGGAGGATTGG |
| | | TGCAGGCTGGGGGCTCTCTGAGACTCTCCTGTGCA |
| | | GCCTCTGGACGCACCTTCAATAGCTTCAACATGGC |
| | | CTGGTTCCGCCAGGCTCCAGGGAAGGCGCGTGAG |
| | | TTTGTAGCAGGTATTATGTGGAGTGGTACGCACAC |
| | | ACGCTATGCAGATTCCGTGAAGGGCCGATTCACCA |
| | | TCTCCAGAGACAACGCCAAGAGCACGGTGCTTCTG |
| | | CAAATGAACAGCCTGAAACCTGAGGACACGGCCGT |
| | | TTATTACTGTGCAGGCCAAATCTATGGCGACTATTT |
| | | CAAGGAGTCCAACATGCAGTACTGGGGCAAAGGG |
| | | ACCCAGGTCACCGTCTCCTCA |
| 38 | CD2A-DNA | CAGGTGCAGCTGCAGGAGTCTGGGGGAGGCTTGG |
| | | TGCAGCCTGGGGGGTCTCTGAGACTCTCCTGTACA |
| | | GCCTCTGGACGCACCTTCAATAGCTTCAACATGGC |
| | | CTGGTTCCGCCAGGGTCCAGGGAAGGCGCGTGAG |
| | | TTTGTAGCAGGTATTATGTGGAGTGGTACACACACA |
| | | CGCTATGCAGATTCCGTGAAGGGCCGATTCACCAT |
| | | CTCCAGAGACAACGCCAAGAGCACGGTGCTTCTGC |
| | | AAATGAACAGCCTGAAACCTGAGGACACGGCCGTT |
| | | TATTACTGTGCAGGCCAAATCTATGGCGACTATTTG |
| | | AAGGAGTCCAACATGCAATACTGGGGCAAAGGGAC |
| | | CCAGGTCACCGTCTCCTCA |
| 39 | CD6E-DNA | CAGGTGCAGCTGCAGGAGTCTGGGGGAGGATTGG |
| | | TGCACACTGGGGGCTCTCTGAGACTCAGCTGTGCA |
| | | GCCTCTGGACGCACCTTCAGTAGCTTCAACATGGC |
| | | CTGGTTCCGCCAGGCTCCAGGGAAGGAGCGTGAG |
| | | TTTGTAGCAGGTATTATGTGGAGTGGTACACACACA |
| | | CGCTATGCAGATTCCGTGAAGGGCCGCGCCACCAT |
| | | CTCCAGAGACAACGCCAAGAACACGGTGCTTCTGC |
| | | AAATGAACAGCCTGAAACCTGAGGACACGGCCGTT |
| | | TATTACTGTGCAGCCCAAATCTACGGCGACTATTTT |
| | | AAGGAGTCCGGCATGCAGTACTGGGGCAAAGGGA |
| | | CCCAGGTCACCGTCTCCTCA |
| 40 | CD2F-DNA | CAGGTGCAGCTGCAGGAGTCTGGGGGAGGCTTGG |
| | | TGCAAACTGGGGGGTCTCTGAGACTCTCCTGTTTA |
| | | GCCTCTGGACGCACCTTCCGTTACTATGCCATGGG |
| | | CTGGTTCCGCCAGGCTCCAGGGAAGGAGCGTGAG |
| | | TTCGTAGCAGGTATTAACATTAGTGGTAGTAACACT |
| | | GACTATTCAGACTCCGTGAAGGGCCGATTCACCAT |
| | | CTCCAAGGACAACGCCAAGAATATGGGGTATCTGC |
| | | AAATGAACAGCCTGAAACCTGAGGACACGGCCGTT |
| | | TATTACTGTGCAGCGAACCGTCGCGGTCCTAATGA |
| | | CTATGAGTACTGGGGCCGGGGGACCCAGGTCACC |
| | | GTCTCCTCA |
| 41 | CD2C-DNA | CAGGTGCAGCTGCAGGAGTCTGGGGGAGGATTGG |
| | | TGCAGGCTGGGGACTCTCTGAGACTCTCCTGTTTA |
| | | GCCTCTGGACGCACCTTCCGTTACTATGCCATGGG |
| | | CTGGTTCCGCCAGGCTCCAGGGAAGGAGCGTGAG |
| | | TTCGTAGCAGGTATTAATATTAGTGGTAGTAACACT |
| | | GACTATTCAGACTCCGTGAAGGGCCGATTCACCAT |
| | | CTCCAAGGACAACGCCAAGAATATGGGGTATCTGC |
| | | AAATGAACAGCCTGAAACCTGAGGACACGGCCGTT |
| | | TATTACTGTGCAGCGAACCGTCGGGGTCGTAATGA |
| | | CTATGAGTACTGGGGCCGGGGGACCCAGGTCACC |
| | | GTCTCCTCA |
| 42 | CD1B-DNA | CAGGTGCAGCTGCAGGAGTCTGGGGGAGGATTGG |
| | | TGCAGGCTGGGGGATCTCTGAGACTCTCCTGTGCA |
| | | GCCTCTGGACGCACCTTCCGTTACTATGCCATGGG |
| | | CTGGTTCCGCCAGGCTCCAGGGAAGGAGCGTGAG |
| | | TTCGTAGCAGGTATTAACATTAGTGGTGGTAACACT |
| | | GACTATCCAGACTCCGTGAAGGGCCGATTCACCAT |
| | | CTCCAGAGACAACGCCAAGAACACGGGGTATCTGC |
| | | AAATGAACAGCCTGAAACCTGAGGACACGGCCGTT |
| | | TATTACTGTGCAGTGAATCGTCGCGGTCAAGATGA |
| | | TTATGAGTACTGGGGCCGGGGGACCCAGGTCACC |
| | | GTCTCCTCA |
| 43 | FIG. 3 CD3A | QVQLQESGGGLVQAGGSLRLSCAASGFTFDDYAMGWFRQAPGK |
| | | EREFVTAISWSGGNTYYADSVKGRFTISRDNAKNTVYLQMNSLKPE |
| | | DTAVYYCAAKPRRTYYSGSDYYTSPYEYDYSGQGTQVTVSSAAASA |
| | | HHHHHH |
| 44 | FIG. 3 CD1C | QVQLQESGGGLVQAGGSLRLSCAASGRTFNSFNMAWFRQAPGK |
| | | AREFVAGIMWSGTHTRYADSVKGRFTISRDNAKSTVLLQMNSLKP |
| | | EDTAVYYCAGQIYGDYFKESNMQYWGKGTQVTVSSAAASAHHH |
| | | HHH |
| 45 | FIG. 3 CD2A | QVQLQESGGGLVQPGGSLRLSCTASGRTFNSFNMAWFRQGPGK |
| | | AREFVAGIMWSGTHTRYADSVKGRFTISRDNAKSTVLLQMNSLKP |
| | | EDTAVYYCAGQIYGDYLKESNMQYWGKGTQVTVSSAAASAHHHH |
| | | HH |
| 46 | FIG. 3 CD6E | QVQLQESGGGLVHTGGSLRLSCAASGRTFSSFNMAWFRQAPGKE |
| | | REFVAGIMWSGTHTRYADSVKGRATISRDNAKNTVLLQMNSLKPE |
| | | DTAVYYCAAQIYGDYFKESGMQYWGKGTQVTVSSAAASAHHHH |
| | | HH |
| 47 | FIG. 3 CD2F | QVQLQESGGGLVQTGGSLRLSCLASGRTFRYYAMGWFRQAPGKE |
| | | REFVAGINISGSNTDYSDSVKGRFTISKDNAKNMGYLQMNSLKPED |
| | | TAVYYCAANRRGPNDYEYWGRGTQVTVSSAAASAHHHHHH |
| 48 | FIG. 3 CD2C | QVQLQESGGGLVQAGDSLRLSCLASGRTFRYYAMGWFRQAPGKE |
| | | REFVAGINISGSNTDYSDSVKGRFTISKDNAKNMGYLQMNSLKPED |
| | | TAVYYCAANRRGRNDYEYWGRGTQVTVSSAAASAHHHHHH |
| 49 | FIG. 3 CD1B | QVQLQESGGGLVQAGGSLRLSCAASGRTFRYYAMGWFRQAPGK |
| | | EREFVAGINISGGNTDYPDSVKGRFTISRDNAKNTGYLQMNSLKPE |
| | | DTAVYYCAVNRRGQDDYEYWGRGTQVTVSSAAASAHHHHHH |
| 50 | FIG. 3 | QVQLQESGGGLVQAGGSLRLSCAASGRTFXXYAMGWFRQAPGK |
| | Consensus | EREFVAGIXWSGXNTXYADSVKGRFTISRDNAKNTVYLQMNSLKP |
| | sequence | EDTAVYYCAAXXRXXYXXXXXXYXYWGXGTQVTVSSAAASAHHHH |
| | | HH |

What is claimed is:

1-46. (canceled)

47. A single domain antibody which binds to Clostridium difficile toxin B, wherein the single domain antibody is selected from the group consisting of:
a) a single domain antibody comprising:
i. a complementary-determining region 1 (CDR1) comprising or consisting of SEQ ID NO: 1; and
ii. a complementary-determining region 2 (CDR2) comprising or consisting of SEQ ID NO: 2; and
iii. a complementary-determining region 3 (CDR3) comprising or consisting of SEQ ID NO: 3;
b) a single domain antibody comprising or consisting of the sequence as set forth in SEQ ID NO: 22, or a sequence having at least 90% sequence identity thereto, wherein any sequence variance is outside the complementary-determining regions (CDRs); and
c) a humanised version of the single domain antibody of a) or b).

48. The single domain antibody according to claim 47, wherein the single domain antibody is able to block at least 20%, such as at least 30%, such as at least 40%, at least 50%, such as at least 60%, such as at least 70%, or such as at least 80% of the enzymatic activity of the glycosyltransferase domain of Clostridium difficile toxin B.

49. The single domain antibody according to claim 48, wherein the glycosyltransferase domain has an amino acid sequence according to SEQ ID NO: 35.

50. The single domain antibody according to claim 47, wherein the single domain antibody comprises a detection label, such as a colorimetric, a fluorescent, a luminescent, a magnetic, or a paramagnetic label, or is biotinylated.

51. The single domain antibody according to claim 47, wherein the single domain antibody comprises or consists of the sequence as set forth in SEQ ID NO: 22.

52. A fusion protein comprising a single domain antibody as defined in claim 47 and one or more further single domain antibodies, and optionally one or more linkers.

53. The fusion protein according to claim 52, wherein the fusion protein is a homodimer or a heterodimer.

54. The fusion protein according to claim 52, wherein the one or more further single domain antibodies bind to Clostridium difficile toxin B and/or Clostridium difficile toxin A.

55. The fusion protein according to claim 52, wherein the linker is a GS linker of the structure (GₓS)ₙ, where x may be a number between 1 to 10, preferably 2 to 5, and n refers to a number of repeats of the GₓS sequence, where n may be between 1 to 10, preferably 2 to 5.

56. The fusion protein according to claim 55, wherein said GS linker is a GGGGS linker (SEQ ID NO: 29), a GGGGSGGGGS linker (SEQ ID NO: 30), a GGGGSGGGGSGGGGS linker (SEQ ID NO: 31), a GGGGGGGGSGGGGSGGGGS linker (SEQ ID NO: 32), a GGGGSGGGGSGGGGSGGGGSGGGGS linker (SEQ ID NO: 33), or a GGGGSGGGGSGGGGSGGGGSGGGGSGGGGS linker (SEQ ID NO: 34).

57. An isolated nucleic acid molecule encoding the single domain antibody according to claim 47.

58. The isolated nucleic acid molecule according to claim 57, wherein the nucleic acid molecule comprises or consists of SEQ ID NO: 36.

59. The isolated nucleic acid molecule according to claim 57, wherein the nucleic acid molecule is codon-optimized for a host cell wherein said nucleic acid molecule is expressed.
